(19) United States
(12) Patent Application Publication
(10) Pub. No.: US 2005/0058603 A1
(43) Pub. Date: Mar. 17, 2005

(54) Drug delivery system based on polymer nanoshells

(75) Inventors: Jinming Gao, Pepper Pike, OH (US); Hua Ai, Cleveland, OH (US)

Correspondence Address: ROPES & GRAY LLP, ONE INTERNATIONAL PLACE, BOSTON, MA, 02110-2624, US

(73) Assignee: Case Western Reserve University, Cleveland, OH (US)

(21) Appl. No.: 10/838,289

(22) Filed: May 3, 2004

Related U.S. Application Data
(60) Provisional application No. 60/502,429, filed on Sep. 12, 2003.
(60) Provisional application No. 60/467,389, filed on May 2, 2003.

(51) Int. Cl. A61K 49/00; A61K 9/16; A61K 9/50

(52) U.S. Cl. 424/9.32; 424/490

(57) ABSTRACT

The present invention relates to polymeric nanoshells. In certain embodiments, the polymeric nanoshells comprise one or more polymeric shells around a hollow core. In other embodiments, the present invention provides nanoshells useful for the delivery of agents such as, for example, various diagnostic and therapeutic agents.

### RELATED APPLICATIONS

This application claims the benefit of priority of U.S. Provisional Application No. 60/467,389 filed on May 2, 2003; and U.S. Provisional Application No. 60/502,429 filed on Sep. 12, 2003. The entire teachings of both applications are hereby incorporated by reference in their entirety.

### FUNDING

Work described herein was funded, in whole or in part, by National Institutes of Health Grant Number R21 CA-93993. The United States government has certain rights in the invention.

### FIELD OF THE INVENTION

The invention relates to the field of microcapsules, and to the fields of sustained-release drug compositions, targeted therapeutics, and medical imaging.

### BACKGROUND OF THE INVENTION

Advanced biomaterials are essential for the successful development of drug delivery systems to achieve a safe and efficacious drug therapy. Effective targeting of both drugs and imaging agents to specific body sites, and precise control of drug release rates, are two primary goals in the management and treatment of localized diseases such as cancer.

Over the last 20 years, despite intense research efforts in academia and industry, only limited success has been achieved in the development of drug carriers that can achieve both goals. Among various challenges, the intrinsic limitations of existing delivery systems (e.g., lack of controlled release of drugs from liposome formulations) and difficulties in characterizing the in vivo pharmacokinetic behavior (e.g., drug targeting efficiency) are among the significant limiting factors.

Drug delivery systems are particularly valuable for improving safety, efficacy and patient compliance in cancer treatment. For an ideal drug delivery system, effective drug targeting to a specific anatomical site and precise control of drug release rates are important to achieve the maximal therapeutic efficacy while minimizing systemic toxicity. Considerable progress has been made in the development of controlled release systems, some of which have led to good clinical outcomes. For example, Lupron Depot™, injectable poly(lactic-co-glycolic acid) microspheres that deliver a growth hormone over a period of 1-4 months, has been used in treating advanced prostate cancer, endometriosis or precocious puberty in more than 300,000 patients, with over $1 billion annual sales. As another example, a polyanhydride surface-eroding polymer is used in an implantable device for delivery of the anticancer drug carmustine (BCNU), for treating glioblastoma multiforme, a malignant brain tumor. This drug delivery device, the Gliadel™ Wafer, was approved in 1996 by the Food and Drug Administration (FDA), making it the first new form of therapy for brain tumors in 25 years.

Drug carriers that actively target drugs to specific body sites are still under intensive investigation. A variety of alternative delivery systems have been developed, including polymer-drug conjugates, immunoliposomes, and polymer nanoparticles. These systems generally show excellent cell targeting and uptake efficiency in vitro, but they are less often successful in in vivo applications. Two primary factors account for the slow clinical progress: (1) numerous physiological barriers (e.g., non-specific uptake by the reticuloendothelial systems, enzyme degradation, decreased binding under flow conditions) present significant challenges for drug targeting upon i.v. administration; and (2) efficient non-invasive methods to evaluate and characterize the targeting efficiency under unperturbed physiological conditions are lacking. There is a need for a drug delivery system that overcomes these obstacles.

Nanoshells hold promise in overcoming these obstacles. For example, ibuprofen microcrystals sized between 5 and 40 microns have been encapsulated with polyelectrolytes, including chitosan, dextran sulfate, carboxymethyl cellulose, and sodium alginate for controlled release (Qiu et al., Langmuir, 2001, 17:5375-5380). The release rate of ibuprofen from the microcapsules decreases as the shell thickness increases. Multilayers of (chitosan/dextran sulfate)₁₀ achieved the longest release time, up to 3 times at pH 7.4 and 4 times at pH 1.4, compared to bare ibuprofen microcrystals. Encapsulation of another drug microcrystal, furosemide, with gelatin A/poly(styrene sulfonate) multilayers resulted in prolonged release up to 300 times longer (Ai et al., J. Control. Release, 2002; 86:59-68).

However, the properties of existing shells limit their biomedical applications. First, most shells have been fabricated from non-biocompatible and/or non-biodegradable synthetic polymers, such as poly(allylamine hydrochloride) (PAH) and poly(styrene sulfonate) (PSS), which present problems in in vivo applications. Second, prolonged drug release in vivo has not clearly been demonstrated using polymers such as PAH, PSS, chitosan, and other materials in shell fabrication. Third, shell sizes have been relatively large, usually on the order of about 5 microns in diameter. In drug delivery applications, a smaller particle diameter (<1 μm) is important for prolonged blood circulation and enhanced drug targeting to specific body sites.

Nanometer-sized shells composed of inorganic particles have been fabricated through high temperature methods, but may not be suitable for clinical applications due to low biocompatibility, lack of controlled release properties, and difficulties in drug encapsulation. For example, U.S. Pat. No. 6,479,146 (incorporated herein by reference) discloses the preparation of hollow silica microspheres via layer-by-layer shell assembly on 640 nm diameter polystyrene latex particles, followed by pyrolysis at 500 ° C. to decompose the polystyrene core. The same assembly procedure was used to prepare silica-containing shells on 3 μm diameter melamine-formaldehyde particles, followed by acid dissolution of the core. There remains a need for methods to fabricate nanometer-sized biocompatible and/or biodegradable shells suitable for use as drug carriers and contrast agents.

### SUMMARY

This invention provides polymeric nanoshells useful for the delivery of bioactive agents such as for example, various diagnostic or therapeutic agents. The invention also provides drug-delivery nanospheres comprising nanoshells loaded with a bioactive agent. In another aspect, the nanoshell is useful for delivering diagnostic agents such as contrast agents or can itself be modified to be useful in diagnostic contexts.

Accordingly, in one embodiment, these nanoshells provide a safe and effective system for targeted drug delivery to specific anatomical sites of interest. This application also provides systems useful for the sustained release of drugs. In yet other aspects, the application provides a means for delivering diagnostic agents such as contrast agents that are useful in generating MRI visibility.

The polymer nanoshells comprise one or more concentric polymeric shells which define a hollow core. The concentric polymeric shells defining the hollow core comprise charged organic polymers, and the diameter of the nanoshells are between 50 and 1000 nanometers in diameter. Organic polymers are polymers consisting of a substantial fraction of carbon, typically 30% or more carbon by weight.

In one embodiment, the nanoshell has an outer surface comprising biocompatible organic polymers. In another aspect, the nanoshell has an outer surface comprising targeting moieties such as for example targeting ligands, peptides, proteins, antibodies, and the like, and a second layer comprising biocompatible organic polymers. In certain embodiments, one or more of the polymeric shells may further comprise superparamagnetic nanoparticles. The polymer nanoshells, which comprise multiple layers of oppositely charged biocompatible organic polymers, may be impregnated with superparamagnetic particles such as iron oxide (SPIO) nanoparticles (˜4 nm) for this purpose.

In an exemplary embodiment, the polymer nanoshell may be used for targeting anticancer agents to a tumor site. These nanoshells may be loaded with any suitable anticancer agent and may be targeted to the tumor using a nanoshell wherein the outer shell comprises tumor-specific antibodies. For example, the anti-Her2/neu antibody may be used to target the nanoshells to breast cancer cells. Such polymer nanoshells can effectively target cancer cells, and if the nanoshell further comprises an MRI contrast agent (e.g., superparamagnetic particles, T1 agents, T2 agents, etc.), cell targeting efficiency can be non-invasively monitored using MRI.

In additional embodiments, the invention relates to polymeric nanoshells comprising a shell surface modified by PEG. In further embodiments, the shell surface comprises PEI25k-PEG5k (1:10).

### BRIEF DESCRIPTION OF THE FIGURES

FIG. 1 is a schematic illustration of an MRI-visible drug-loaded nanoshell.

FIG. 2 illustrates the layer-by-layer (LbL) assembly of polymers and biomolecules on a flat substrate.

FIG. 3A shows MF nanoparticle diameter as a function of time and pH after suspension in aqueous acid.

FIG. 3B shows scanning electron microscopy (SEM) images of MF core particles at various degrees of surface erosion in pH 2.0 HCl solution. Panels A and D show the original particles, prior to hydrolysis. Panels B and E show particles after 8 minutes' hydrolysis, and Panels C and F show particles after 60 minutes' hydrolysis.

FIG. 4 shows micrographs of nanoshells composed of (gelatin/PDDA)₅ multilayers. Panel A: 1.2 micron diameter shells; Panel B: 600 nm diameter shells; Panel C: 390 nm diameter shells. The inserts show individual shells at higher magnification.

FIG. 5 shows tapping mode SFM images of 620 nm nanoshells. Panel A: SFM image of nanoshells adsorbed on a mica substrate. Panel B: 3-D tapping mode SFM image of an individual 620 nm nanoshell. The thickness of the ring is about 30 nm.

FIG. 6 shows confocal microscopy images of 5 μm polymer shells. Panel A: shells at pH 3.0 during drug loading process with DOX; Panel B: DOX loaded shells at pH 7.4. The scale bars are 10 μm in both images.

FIG. 7 is a fluorescence micrograph of 5 μm polymer shells composed of (gelatin/PLL)₅, with a surface coating of albumin-FITC conjugate.

FIG. 8 is a schematic illustration of the process for preparing dual-function polymer nanoshells.

FIG. 9 shows a scheme for fabrication of 4 nm SPIO nanoparticles, and chemical modification of the particles' surface.

FIG. 10 is a schematic illustration of layer-by-layer fabrication of nano-organized shells.

FIG. 11 shows SEM images of nanoshells.

FIG. 12 shows confocal images of shells composed of (gelatin/PDDA)₅ and silica and PDDA and lipid bilayers.

FIG. 13 is a graph depicting the results of the cytotoxicity study of doxorubicin loaded shells compared to doxorubicin solution and empty shells.

FIG. 14 shows optical and confocal images of shell-cell interactions. Panels A, B, C, and D indicate the same focal plane and the same field under bright field, confocal nuclei, confocal polymer shells, and the combined figure, respectively. Shells were clearly attached to the membrane. Panels E, F, G, and H use the same sequence as A, B, C, and D. One shell was located inside the cell but outside the nucleus.

FIG. 15 is a schematic illustration of self-assembly of hollow polyelectrolyte shells.

FIG. 16 shows shell characterization by SEM and CLSM. A. SEM figure of 1μ MF particles covered with [(gelatin/PDDA)₅+(nanoparticle/PDDA)]. B. Amplified image of a shell before core dissolution. 50 nm Fluoresbrite® YG Carboxylate nanoparticles were clearly seen on the shell surface. C. SEM figure of 1μ hollow shells after core dissolution in acid. D. Confocal images of hollow shells dispersed in PBS.

FIG. 17 shows shell surface charge before and after culture media incubation.

FIG. 18 shows flow cytometry data of shell uptake at different time points. Shells covered with PEI were incubated with human breast cancer cell line MCF-7. More cells have internalized shells with longer period of incubation time. M2 region represents the percentage of cells with internalized shells (30 min: 18.9%; 2 hr: 43.6%; 4 hr: 51.7%).

FIG. 19 shows cell uptake of shells with positive or negative charges. PDDA displays the highest cell uptake percentage of 52.6±4.4% and 70.3±1.6% at 4 and 24 hours respectively. Lipid bilayers present the highest cell uptake among all the formulations with the percentage of 78.7±2.5% at 24 hours time point.

FIG. 20 shows shell surface property and cell uptake of shells covered with PEI-PEG copolymer or PEI. A: PEI-PEG copolymer covered shells do not show dramatic surface charge change compared to PEI polymer coated shells after one-hour cell culture media incubation. B: PEI24k-PEG5k (1:10) copolymer covered shells have the lowest cell uptake compared to other copolymer groups and PEI group (p<0.01).

FIG. 21 shows CLSM study of shell-cell interactions. A, B, and C are 1-micron shells with MCF-7 cells. A: cell nuclei are labeled. B: cell membranes are labeled and shells are internalized. C: registration of A and B.

### DETAILED DESCRIPTION OF THE INVENTION

The present invention provides nanoshells having sub-micrometer diameters, methods of making them, and methods of using them to deliver bioactive agents for therapeutic and diagnostic purposes. The nanoshells of the invention are preferably composed of biocompatible organic polymers, which are most preferably biodegradable as well. They may be fabricated upon the surface of nanoparticle cores through an electrostatic layer-by-layer (LbL) self-assembly technique, which permits precise control of the diameter (e.g., 100, 300, or 600 nm) and thickness (e.g., 10 nm or 30 nm) of the shells. In this method, alternating layers of positively and negatively charged organic polymers are laid down upon the core particles until a shell having the desired number of layers is obtained. The number of individual layers may range from two to thirty or more, depending on the size, thickness, and release properties desired. Removal of the core, typically via chemical decomposition, provides the nanoshells of the invention.

The nanoshells of the invention provide an improved system for targeted and/or controlled release drug delivery applications, and for localization of diagnostic imaging reagents. More specifically, therapeutic agent may be enclosed within the nanoshells so as to form nanospheres, which provide controlled release of the encapsulated therapeutic agent. The nanoshells of the invention may optionally incorporate targeting moieties displayed on their outer surfaces, so as to provide effective drug targeting to specific organs or tissues, and they may also incorporate diagnostic agents, such as contrast agents for imaging the targeted organs or tissues.

Assembly of the nanoshells typically begins with a suspension of nanoparticulate cores, such as a colloidal suspension of polymeric nanoparticles. The nanoparticle cores may be constructed of any solid material that has (or can be given) a surface charge, and which can be dissolved after the shell layers have been formed without disrupting the layered shell coating. Suitable core materials include but are not limited to melamine formaldehyde, poly(lactic acid-co-lysine), amino- and carboxy-substituted polycarbonates, polyesters, polyacetals, polyacrylates, and polystyrenes, and various copolymers thereof, as well as inorganic core materials such as colloidal silica, titania, or zirconia, or finely divided metallic oxides and carbonates such as MnCO₃ microcrystals. For example, commercially available monodisperse polystyrene, poly(methyl methacrylate), or melamine formaldehyde particles ranging from 1 to 5 μm in diameter, may be used as cores for hollow nanoshell formation. If necessary, the particles are reduced in size by an appropriate means, for example by partial dissolution, decomposition, or erosion, before they are used as cores for nanoshell fabrication. Surface charges, if not already present in the cores, may be introduced by methods known in the art, for example by coating with a layer of charged polymer, or by surface oxidation and/or coupling of charged chemical moieties. See for example Surface-Controlled Nanoscale Materials for High-Added-Value Applications, K. E. Gonsalves et al., Eds, 1998, Materials Research Society (Warrendale, Pa.), and Synthesis, Functionalization and Surface Treatment of Nanoparticles, M.-I. Baraton, Ed., 2003, American Scientific Publishers (Stevenson Ranch, Calif.).

In one embodiment, monodisperse melamine formaldehyde (MF) nanoparticles are prepared as core materials for shell assembly. Using this method, it is possible to assemble monodisperse nanoshells ranging from 90 to 1000 nm in diameter, or even 50 nm to 1000 nm in diameter based on MF templates. By way of example, the preparation of 300 nm and 600 nm polymer shells are described in detail herein.

The shell materials consist largely or entirely of ionic or amphoteric polymers, preferably organic polymers, which are preferably biocompatible and most preferably are biodegradable as well. The shell layers can comprise polyanions, polycations, charged biopolymers, and lipid bilayers. Suitable materials include but are not limited to gelatin, chitosan, dextran sulfate, carboxymethyl cellulose, sodium alginate, poly(styrene sulfonate) (PSS), poly(lysine), poly(acrylic acid), poly(dimethyldiallyl ammonium chloride) (PDDA), and poly(allylamine hydrochloride) (PAH). Particularly versatile are polyelectrolytes, and amphoteric organic polymers such as gelatin which may be given a positive or negative charge by varying the pH of the environment, and thus may be coated upon, or coated with, either a polycation or polyanion. Any or all of the layers may independently comprise or consist of biocompatible and/or bioerodable materials. The identification and selection of appropriate materials is well within the ability of those skilled in the art. In general, biocompatible layer materials are those which do not provoke an immune or inflammatory reaction, and which do not exhibit either local or systemic toxicity. Bioerodable layer materials are those which, after administration, are degraded in vivo, through enzymatic action and/or as a consequence of non-enzyrnatic hydrolysis, into non-toxic products that are subject to catabolism, metabolism, or excretion.

In certain embodiments, the polymeric materials are useful in prolonging drug release. For example, using gelatin in the shell assembly has been found to prolong drug release significantly. In alternative embodiments, albumin may be added as the outermost layer of the polymeric shell.

In one embodiment, the nanoshells are composed of multilayers of PDDA and gelatin. For biocompatibility considerations, PDDA may be replaced with cationic poly-L-lysine (PLL). Nanoshells fabricated from gelatin and PLL are both biocompatible and biodegradable. In preferred embodiments, therefore, the nanoshells are composed of biocompatible organic polymers, assembled by the electrostatic layer-by-layer (LbL) method. The shell diameter may be between 100 and 1500 nanometers, and is preferably between 100 and 600 nm. The shell thickness may be between 10 and 100 nm, preferably between 10 and 30 nm. Drug release kinetics may be varied by varying the nanoshell membrane properties (e.g., thickness, polymer identities, polymer molecular weights, and additives).

Construction of polyelectrolyte nanoshells involves colloid-templated consecutive polyelectrolyte adsorption on a nanosphere core, followed by decomposition of the core material. The use of polyelectrolytes in layer-by-layer assembly methods has been described previously; see for example Handbook of Polyelectrolytes and Their Applications, Vol. I: Polyelectrolyte-Based Multilayers, Self-Assemblies and Nanostructures, S. Tripathy et al., Eds., 2002, American Scientific Publishers (Stevenson Ranch, Calif.).

LbL self-assembly is a versatile technique that has been applied in thin film coating, micropatterning, nanobioreactors, artificial cells, drug delivery systems, and electronic devices. The LbL technique is based on alternate adsorption of oppositely charged materials, such as linear polycations and polyanions. Multilayers of materials can be assembled on two-dimensional (2-D) supports of any area (slides, silicon wafers, plastic surfaces) and on 3-dimensional (3-D) micro/nanotemplates (e.g., colloidal particles, such as latex or cells). Ultrathin ordered films can be designed with molecular architecture plans in the range of 5 to 1000 nm, with a precision better than 1 nm and a definite knowledge of their molecular composition. Charged materials, including linear polyelectrolytes (synthetic and natural), enzymes, antibodies, viruses and inorganic nanoparticles have been used in 2-D and 3-D nanoassembly processes. The architecture of the resulting film can be designed with nanometer precision (in cross-section) to meet different requirements such as thickness, biocompatibility, controlled permeability, targeting, and optical or magnetic properties.

In some embodiments, the outermost shell further comprises targeting moieties such as proteins, peptides, ligands, and antibodies. Suitable targeting moieties include, but are not limited to, peptides such as homing peptides, proteins, receptor-specific ligands and tissue-specific antibodies (e.g., tumor-specific antibodies, such as anti-Her2/neu).

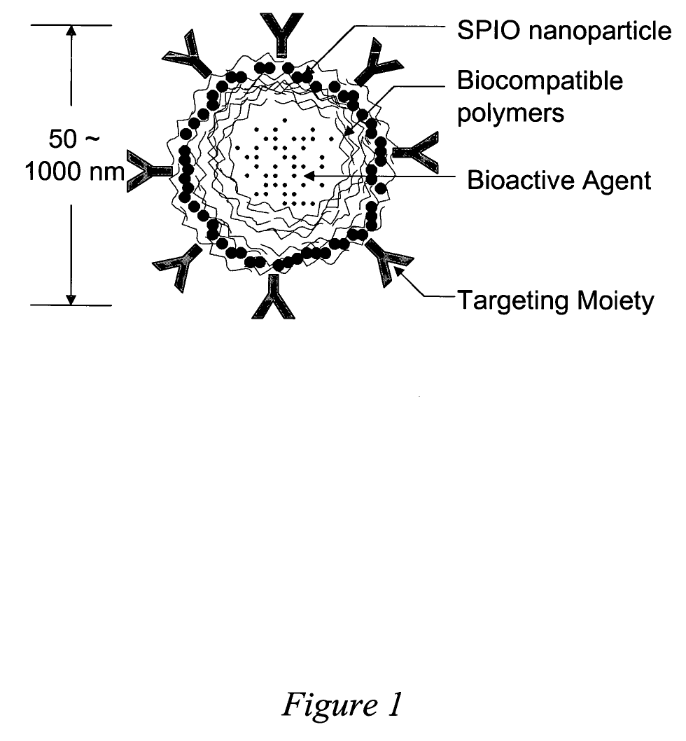

In one particular embodiment, shown in FIG. 1, the nanoshell comprises one or more of the following:

(1) an outer shell functionalized with targeting moieties;

(2) a nanoshell membrane comprising a plurality of layers of biocompatible organic polymers and, optionally, a diagnostic agent such as a contrast agent; and

(3) an interior region, defined by the nanoshell membrane, that may be loaded with a bioactive agent such as a therapeutic or diagnostic agent.

In certain embodiments, such as that shown in FIG. 1, the nanoshell further comprises a magnetic imaging contrast agent, such as a T1 or T2 contrast agent, preferably superparamagnetic nanoparticles such as the SPIO nanoparticles shown in FIG. 1. Incorporating such superparamagnetic nanoparticles into the shell renders the shell assembly visible to magnetic resonance imaging. These nanoshells can exploit non-invasive MRI imaging techniques to provide pharmacokinetic data under unperturbed physiological conditions, which can be used to facilitate the design and monitor the use of targeted drug delivery systems.

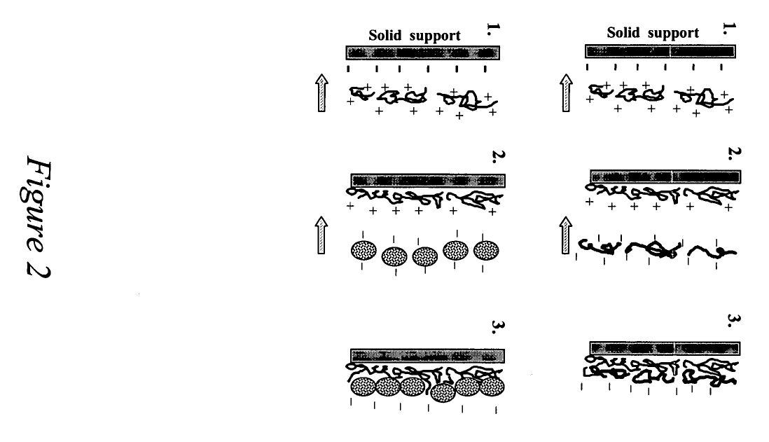

FIG. 2 shows the general self-assembly procedure for polymers (upper scheme) and biomolecules such as enzymes (lower scheme). A solid support (e.g., a glass slide) having a negative surface charge is incubated in the solution containing the cationic polyelectrolytes, and a layer of polycation is then adsorbed (step 1). Because the adsorption is carried out at a relatively high concentration of polyelectrolytes, a number of ionic groups remain exposed at the interface with the solution, and thus the surface charge is effectively reversed. The reversed surface charge prevents further polyion adsorption. The solid support is then rinsed with water to remove excess free polyions. The surface is then immersed in a solution of anionic polyelectrolytes (upper scheme) or enzymes (lower scheme) (step 2). Again a layer is adsorbed, but now the original surface charge (negative) is restored and the surface is ready for further assembly (step 3). These two steps are repeated alternately until a layer of the desired thickness is obtained. More than two components can be used in the assembly, so long as there is an alternation of positively and negatively charged materials.

In one embodiment of the invention, melamine formaldehyde (MF) colloidal particles are used as templates and multiple layers of polyelectrolytes are coated on the surface. After each coating step, the excess polyelectrolytes in solution are typically washed away before the next layer is deposited. After the desired polyelectrolyte layers are deposited, the core of the coated particles is decomposed by an appropriate treatment. For example, MF cores may be decomposed by exposure of the coated particles to a sulfite salt, or to a hydrochloric acid solution at pH 1. After core decomposition, hollow shells may be obtained upon washing.

The shell thickness can be precisely controlled through the number of coated layers. Where hollow shells are intended for drug or enzyme delivery, understanding and controlling the shell permeability is important in membrane design. Permeability of small molecules can be measured indirectly through a 2-D diffusion model or by means of fluorescence recovery following photobleaching.

In order to reduce protein binding, immune system recognition, nonspecific uptake by the reticuloendothelial system, and enzymatic degradation, the outermost layer may optionally display a surface incorporating masking moieties, such as for example poly(ethylene glycol) moieties or serum albumin. See for example M. Akerman et al., Proc. Natl. Acad. Sci. U.S.A. 99:12617-21 (2002). In order to facilitate visualization of the distribution of the nanospheres within the body, one or more of the layers may also optionally incorporate an imaging moiety, such as for example magnetic nanoparticles, a radioisotope, or a radio-opacifying moiety.

In order to obtain more advantageous distribution of the nanoshells or nanospheres in the body, (e.g., for diagnostic imaging purposes or to improve the tissue selectivity of drug delivery), tissue-targeting moieties may be incorporated into the outermost shell. Examples of targeting moieties include but are not limited to lipoproteins, glycoproteins, asialoglycoproteins, transferrin, toxins, carbohydrates, cell surface receptor ligands, antibodies, and homing peptides. Synthetic homing peptides with the desired levels of affinity and/or selectivity for specific organs or tissues may be employed as targeting moieties, for example as disclosed in U.S. Pat. Nos. 6,576,239, 6,306,365, 6,303,573, 6,296,832, 6,232,287, 6,180,084, 6,174,687, 6,068,829, and 5,622,699, U.S. patent applications 2001/0046498, 2002/0041898, 2003/0008819, and 2003/0077826, and PCT application PCT/GB02/04017 (WO 03/020751), all of which are incorporated herein by reference.

Methods for identifying and using these and other tissue-homing peptides are known in the art, see for example W. Arap et al., Science 279:377-380 (1998); R. Pasqualini, and E. Ruoslahti, Nature 380:364-366 (1996); D. Rajotte et al., J. Clin. Invest. 102:430-437 (1998); P. Laakkonen et al., Nature Medicine 8(7):751-755 (2002); and K. Essler, E. Ruoslahti, Proc. Natl. Acad. Sci. U.S.A. 99(4):2252-2257 (2002). The nanoshells of the present invention are in the size range of the filamentous phage typically used for in vivo panning of phage-displayed peptide libraries (fd phage, for example, are about 800 nm in length). Homing peptides identified by in vivo panning, which are capable of binding phage particles to specific tissues, are therefore expected to bind the nanoshells of the present invention to the same tissues, with similar specificity. Suitable tissue-specific homing peptides include but are not limited to the following:

Brain:

| | |
|---|---|
| CLSSRLDAC | CVLRGGRC |
| CNSRLQLRC | CGVRLGC |
| CKDWGRIC | CLDWGRIC |
| CTRITESC | CETLPAC |
| CRTGTLFC | CGRSLDAC |
| CRHWFDVVC | CANAQSHC |
| CGNPSYRC | WRCVLREGPAGGCAWFNRHRL |
| YPCGGEAVAGVSSVRTMCSE | LNCDYQGTNPATSVSVPCTV |
| CNSRLHLRCCENWWGDVC | WRCVLREGPAGGCAWFNRHRL |

Kidney:

| | |
|---|---|
| CLPVASC | CGAREMC |
| CKGRSSAC | CWARAQGC |
| CLGRSSVC | CTSPGGSC |
| CMGRWRLC | CVGECGGC |
| CVAWLNC | CRRFQDC |
| CLMGVHC | CKLLSGVC |
| CFVGHDLC | CRCLNVC |
| CKLMGEC | |

Heart:

| | |
|---|---|
| GGGVFWQ | HGRVRLPH |
| VVLVTSS | CLHRGNSC |
| CRSWNKADNRSC | |

Gut:

| | |
|---|---|
| YAGFFLV | RSGARSS |
| CVESTVA | SRRQPLS |
| SKVWLLL | QVRRVPE |
| YSGKWGW | MVQSVG |
| LRAVGRA | MSPQLAT |
| GAVLPGE | WIEEAER |
| LVSEQLR | RGDRPPY |
| VRRGSPQ | RVRGPER |
| GISAVLS | GGRGSWE |
| GVSASDW | FRVRGSP |
| SRLSGGT | WELVARS |
| MRRDEQR | GCRCWA |
| LSPPYMW | LCTAMTE |

Integrins:

| | |
|---|---|
| CRGDC | CRGDCL |
| CRGDCA | NGRAHA |
| DGRAHA | RCDVVV |
| SLIDIP | TIRSVD |
| KRGD | RRGD |
| RGDL | |

RGD-binding determinants:

| | |
|---|---|
| CSFGRGDIRNC | CSFGRTDQRIC |
| CSFGKGDNRIC | CSFGRNDSRNC |
| CSFGRVDDRNC | CSFGRADRRNC |
| CSFGRSVDRNC | CSFGKRDMRNC |
| CSFGRWDARNC | CSFGRQDVRNC |
| CSFGRDDGRNC | |

Angiogenic tumor endothelium:
CDCRGDCFC
CNGRCVSGCAGRC

Ovary:

| | |
|---|---|
| EVRSRLS | RVGLVAR |
| AVKDYFR | GVRTSIW |
| RPVGMRK | RVRLVNL |
| FFAAVRS | KLVNSSW |
| LCERVWR | FGSQAFV |
| WLERPEY | GGDVMWR |
| VRARLMS | TLRESGP |

Uterus:
GLSGGRS
SWCEPGWCR

Prostate:

| | |
|---|---|
| EVQSAKW | KRVYVLG |
| GRLSVQV | WKPASLS |
| FAVRVVG | LVRPLEG |
| GFYRMLG | EGRPMVY |
| GSRSLGA | RVWQGDV |
| GDELLA | FVWLVGS |
| GSEPMFR | VSFLEYR |
| WHQPL | SMSIARL |
| RGRWLAL | QVEEFPC |
| LWLSGNW | GPMLSVM |
| WTFLERL | VLPGGQW |
| REVKES | RTPAAVM |
| GEWLGEC | PNPLMPL |
| SLWYLGA | YVGGWEL |

Lung:

| | |
|---|---|
| CGFECVRQCPERC | CTLRDRNC |
| CIKGNVNC | CRHESSSC |
| CLYIDRRC | CYSLGADC |
| CSKLMMTC | CGFELETC |
| CNSDVDLC | CVGNLSMC |
| CEKKLLYC | CKGQRDFC |
| CTFRNASC | CNMGLTRC |
| CHEGYLTC | CGTFGARC |
| CIGEVEVC | CRISAHPC |
| CLRPYLNC | CSYPKILC |
| CMELSKQC | CSEPSGTC |
| CGNETLRG | CTLSNRFC |
| CMGSEYWC | CLFSDENC |
| CAHQHIQC | CKGQGDWC |
| CAQNMLCC | CWRGDRKIC |
| CLAKENVVC | CIFREANVC |
| CRTHGYQGC | CERVVGSSC |
| CKTNHMESC | CYEEKSQSC |
| CKDSAMTIC | CTRSTNTGC |
| CMSWDAVSC | CKWSRLHSC |
| CMSPQRSDC | CLHSPRSKC |
| CPQDIRRNC | CLYTKEQRC |
| CQTRNFAQC | CTGHLSTDC |
| CQDLNIMQC | TRRTNNPLT |
| CGYIDPNRISQC | CTVNEAYKTRMC |
| CRLRSYGTLSLC | CAGTCATGCNGVC |
| CADYDLALGLMC | CPKARPAPQYKC |
| CSSHQGGFQHGC | CQETRTEGRKKC |
| CRPWHNQAHTEC | CSFGTHDTEPHC |
| CSEAASRMIGVC | CWEEHPSIKWWC |
| CWDADQIEGIKC | CVDSQSMKGLVC |
| CRLQTMGQGQSC | CRPAQRDAGTSC |
| CGGRDRGTYGPC | CGEVASNERIQC |
| CNSKSSAELEKC | CVLNFKNQARDC |
| CRGKPLANFEDC | CEGHSMRGYGLC |
| CRDRGDRMKSLC | CDNTCTYGVDDC |
| CSAHSQEMNVNC | CGAACGVGCRGRC |
| CGFECVRQCPERC | CLVGCRLSCGGEC |
| CRSGCVEGCGGRC | CIARCGGACGRHC |
| CGGECGWECEVSC | CGVGCPGLCGGAC |
| CKWLCLLLCAVAC | CSEGCGPVCWPEC |
| CGAACGVGCGGRC | CSGSCRRGCGIDC |
| CGASCALGCRAYC | CDTSCENNCQGPC |
| CSRQCRGACGQPC | CYWWCDGVCALQC |
| CAGGCAVRCGGTC | CGGACGGVCTGGC |
| CGRPCVGECRMGC | CLVGCEVGGSPAC |
| CPRTCGAACASPC | CRGDCGIGCRRLC |
| CCFTNFDCYLGC | |

Skin:

| | |
|---|---|
| CYADCEGTCGMVC | CWNICPGGCRALC |
| GPGCEEECQPAC | CKGTCVLGCSEEC |
| CSTLCGLRCMGTC | CMPRCGVNCKWAC |
| CVGACDLKCTGGC | CVALCREACGEGC |
| CSSGCSKNCLEMC | CGRPCRGGCAASC |
| CQGGCGVSCPIFC | CAVRCDGSCVPEC |
| CGFGCSGSCQMQC | CRVVCADGCRFIC |
| CTMGCTAGCAFAC | CEGKCGLTCECTC |
| CNQGCSGSCDVMC | CASGCSESCYVGC |
| CGGGCQWGCAGEC | CSVRGKSVCIGLC |
| CPSNCVALCTSGC | CVEGCSSGGGPGC |
| CRVVCADGCRLIC | CSTLCGLRCMGTC |
| CFTFCEYHCQLTC | |

Retina:

| | |
|---|---|
| CRRIWYAVC | CSAYTTSPC |
| CSCFRDVCC | CTDKSWPC |
| CTDNRVGS | CTIADFPC |
| CTSDISWWDYKC | CTVDNELC |
| CVGDCIGSCWMFC | CVKFTYDC |
| CVSGHLNC | CYGESQQMC |
| CYTGETWTC | CAVSIPRC |
| CDCRGDCFC | CDSLCGGACAARC |
| CERSQSKGVHHC | CFKSTLLC |
| CFWHNRAC | CGDVCPSECPGWC |
| CGEFKVGC | CGLDCLGDCSGAC |
| CGPGYQAQCSLRC | CGSHCGQLCKSLC |
| CHMGCVSPCAYVC | CILSYDNPC |
| CISRPYFC | CKERLEYTRGVC |
| CKERPSNGLSAC | CKPFRTEC |
| CKSGCGVACRHMC | CLKPGGQEC |
| CMDSQSSC | CMNILSGC |
| CNIPVTTPIFGC | CNQRTNRESGNC |
| CNRKNSNEQRAC | CNRMEMPC |
| CQIRPIDKC | CAIDIGGAC |
| CGRFDTAPQRGC | CKRANRLSC |
| CLLNYTYC | CLNGLVSMC |
| CMSLGNNC | CNRNRMTPC |
| CQASASDHC | CQLINSSPC |
| CQRVNSVENASC | CRKEHYPC |
| CRRHMERC | CSGRPFKYC |
| CTHLVTLC | CTSSPAYNC |
| CVTSNLRVC | CWDSGSHIC |
| CERSHGRLC | CGNLLTRRC |
| CINCLSQC | CLRHDFYVC |
| CNSRSENC | CRYKGPSC |
| CSHHDTNC | CSRWYTTC |
| CYAGSPLC | CQTTSWNC |
| CQWSMNVC | CRARIRAEDISC |
| CRDVVSVIC | CRREYSAC |

Pancreas:

| | |
|---|---|
| EICQLGSCT | WRCEGFNCQ |
| RKCLRPDCG | SWCEPGWCR |
| LACFVTGCL | GLCNGATCM |
| DMCWLIGCG | SGCRTMVGV |
| QRCPRSFCL | LSCAPVICG |
| RECTNEICY | NECLMISCR |
| SCVFCDWLS | WACEELSCF |
| QNCPVTRCV | CATLTNDEC |
| CDNREMSC | CFMDHSNC |
| CGEYGREC | CHMKRDRTC |
| CKKRLLNVC | CLDYHPKC |
| CMTGRVTC | CNKIVRRC |
| CPDLLVAC | CSDTQSIGC |
| CSKAYDLAG | CSKKGPSYC |
| CTLKHTAMC | CTQHIANC |
| CTTEIDYC | CVGRSGELC |

Liver

| | |
|---|---|
| ARRGWTL | SRRFVGG |
| QLTGGCL | ALERRSL |
| KAYFRWR | RWLAWTV |
| VGSFIYS | LSLLGIA |
| LSTVLWF | SLAMRDS |
| GRSSLAC | SELLGDA |
| CGGAGAR | WRQNMPL |
| DFLRCRV | QAGLRCH |
| RALYDAL | WVSVLGF |
| GMAVSSW | SWFFLVA |
| WQSVVRV | VKSVCRT |
| CGNGHSG | AEMEGRD |
| SLRPDNG | PAMGLIR |

Lypmph Node:

| | |
|---|---|
| WGCKLRFCS | MECIKYSCL |
| GICATVKCS | PRCQLWACT |
| TTCMSQLCL | SHCPMASLC |
| GCVRRLLCN | TSCRLFSCA |
| KYCTPVECL | RGCNGSRCS |
| MCPQRNCL | PECEGVSCI |
| AGCSVTVCG | IPCYWESCR |
| GSCSMFPCS | QDCVKRPCV |
| SECAYRACS | WSCARPLCG |
| SLCGSDGCR | RLCPSSPCT |
| MRCQFSGCT | RYCYPDGCL |
| STCGNWTCR | LPCTGASCP |
| CSCTGQLCR | LECRRWRCD |
| GLCQIDECR | TACKVAACH |
| DRCLDIWCL | XXXQGSPCL |
| PLCMATRCA | RDCSHRSCE |
| NPCLRAACI | PTCAYGWCA |
| LECVANLCT | RKCGEEVCT |
| EPCTWNACL | LVCPGTACV |
| LYCLDASCL | ERCPMAKCY |
| LVCQGSPCL | QQCQDPYCL |
| DXCXDIWCL | QPCRSMVCA |
| KTCVGVRV | WSCHEFNCR |
| LTCWDWSCR | SLCRLSTCS |
| KTCAGSSCI | VICTGRQCG |
| NPCFGLLV | SLCTAFNCH |
| RTCTPSRCM | QSCLWRICI |
| QYCWSKGCR | LGCFPSWCG |
| VTCSSEWCL | RLCSWGGCA |
| STCISVHCS | EVCLVLSCQ |
| IACDGYLCG | RDCVKNLCR |
| XGCYQKRCT | LGCFXSWCG |
| IRCWGGRCS | IPCSLLGCA |
| AGCVQSQCY | PRCWERVCS |
| KACFGADCX | TLCPLVACE |
| SACWLSNCA | SECYTGSCP |
| GLCQEHRCW | VECGFSAVF |
| EDCREWGCR | HWCRLLACR |

Adrenal Gland:

| | |
|---|---|
| WGCKLRFCS | MECIKYSCL |
| GICATVKCS | PRCQLWACT |
| TTCMSQLCL | SHCPMASLC |
| GCVRRLLCN | TSCRLFSCA |
| KYCTPVECL | RGCNGSRCS |
| MCPQRNCL | PECEGVSCI |
| AGCSVTVCG | IPCYWESCR |
| GSCSMFPCS | QDCVKRPCV |
| SECAYRACS | WSCARPLCG |
| SLCGSDGCR | RLCPSSPCT |
| MRCQFSGCT | RYCYPDGCL |
| STCGNWTCR | LPCTGASCP |
| CSCTGQLCR | LECRRWRCD |
| GLCQIDECR | TACKVAACH |
| DRCLDIWCL | XXXQGSPCL |
| PLCMATRCA | RDGSHRSCE |
| NPCLRAACI | PTCAYGWCA |
| LECVANLCT | RKCGEEVCT |
| EPCTWNACL | LVCPGTACV |
| LYCLDASCL | ERCPMAKCY |
| LVCQGSPGL | QQCQDPYCL |
| DXCXDIWCL | QPCRSMVCA |
| KTCVGVRV | WSCHEFNCR |
| LTCWDWSCR | SLCRLSTCS |
| KTCAGSSCI | VICTGRQCG |
| NPCFGLLV | SLCTAFNCH |
| RTCTPSRCM | QSCLWRICI |
| QYCWSKGCR | LGCFPSWCG |
| VTCSSEWCL | RLCSWGGCA |
| STGISVHCS | EVCLVLSCQ |
| IACDGYLCG | RDCVKNLCR |
| XGCYQKRCT | LGCFXSWCG |
| IRCWGGRCS | IPGSLLGCA |
| AGCVQSQCY | PRCWERVCS |
| KACFGADCX | TLCPLVACE |
| SAGWLSNGA | SECYTGSCP |
| GLCQEHRCW | VECGFSAVF |
| EDCREWGCR | HWCRLLACR |

In addition, peptides that may be useful for targeting the nanoshells of the present invention to tumors in vivo include but are not limited to the peptide sequences shown in Table 1, which have been described as potential targeting peptides for tumor cells:

TABLE 1

| | | |
|---|---|---|
| CGRECPRLCQSSC | CGEACGGQCALPC | PSCAYMCIT |
| SKVLYYNWE | CERACRNLCREGC | CKVCNGRCCG |
| CPTCNGRCVR | CRNCNGRCEG | CTECNGRCQL |
| CAVCNGRCGF | CWGCNGRCRM | CVPCNGRCHE |
| CVQCNGRCAL | CGRCNGRCLL | CVWCNGRCGL |
| CEGVNGRRLR | CGSLVRC | SKGLRHR |
| KMGPKVW | NPRWFWD | SGWCYRC |
| CWSGVDC | IVADYQR | LSMFTRP |
| CVMVRDGDC | CGVGSSC | CGEGHPC |
| CPEHRSLVC | CWRKFYC | CPRGSRC |
| CAQLLQVSC | CTDYVRC | TDCTPSRCT |
| CTAMRNTDC | VTCRSLMCQ | CISLDRSC |
| CYLVNVDC | RHCFSQWCS | EACEMAGCL |
| QWCSRRWCT | NACESAICG | FPCEGKKCL |
| AGCINGLCG | KGCGTRQCW | KRCSSSLCA |
| LDCLSELCS | IYCPGQECE | EDCTSRECS |
| RWCREKSCW | CNKTDGDEGVTC | CPLCNGRCAL |
| CEQGNGRCGQ | CVTCNGRCRV | CETCNGRCVG |
| CSCCNGRCGD | CKSCNGRCLA | CRTCNGRCQV |
| CASNNGRVVL | CSKCNGRCGH | CGECNGRCVE |
| CEVCNGRCAL | HHTRFVS | WRVLAAF |
| SPGSWTW | IKARASP | LWAEMTG |
| SKSSGVS | VVDRFPD | IMYPGWL |
| CQLAAVC | CGLSDSC | CELSLISKC |
| CYVELHC | CYSYFLAC | CDDSWKC |
| CKALSQAC | VPCRFKQCW | CMEMGVKC |
| CGTRVDHC | CYLGVSNC | LVCLPPSCE |
| ISCAVDACL | RSCIKHQCP | GICKDLWCQ |
| NRCRGVSCT | FGCVMASCR | DTCRALRCN |
| YRCIARECE | QACPMLLCM | HTCLVALCA |
| RKCEVPGCQ | EICVDGLCV | RLPCGDQACE |
| CEMCNGRCMG | CGVCNGRCGL | CVLCNGRCWS |
| CRTCNGRCLE | CRDLNGRKVM | CPLCNGRCAR |
| CQSCNGRCVR | CRCCNGRCSP | CWLCNGRCGR |
| CIRCNGRCSV | CLSCNGRCPS | GRSQMQI |
| VASVSVA | IFSGSRE | GRWYKWA |
| ALVGLMR | DTLRLRI | VWRTGHL |
| GLPVKWS | CVRIRPC | CVSGPRC |
| CYTADPC | CLVVHEAAC | CFWPNRC |
| CRLGIAC | CYPADPC | CGETMRC |
| SWCQFEKCL | CRESLKNC | CNNVGSYC |
| CAMYSMED | CIRSAVSC | FYCPGVGCR |
| PRCESQLCP | MFCRMRSCD | APCGLLACI |
| ADCRQKLPCL | RSCAEPWCY | GRCVDGGCT |
| ICLLAHCA | AGCRVESC | RLCSLYGCV |
| LECVVDSCR | FRCLERVCT | CNGRCVSGCAGRC |
| IWSGYGVYW | WESLYFPRE | CGLMCQGACFDVC |
| CPRGGLAVCVSQG | RLCRIVVIRVCR | |

YVPLPNVPQPGRRPFPTFPGQGPFNPKIKWPQGY
VFIDILDKVENAIHNAAQVGIGFAKPFEKLINPK
GNNRPVYIPQPRPPHPRI
GNNRPVYIPQPRPPHPRL
GNNRPIYIPQPRPPHPRL
RFRPPIRRPPIRPPFYPPFRPPIRPPIFPPIRPPFRPPLRFP
RRIRPRPPRLPRPRPRPLPFPRPGPRPIPRPLPFPRPGPRPIPRPLPFFR
PGPRPIPRP
PRPIPRPLPFFRPGPRPIPR
WNPFKELERAGQRVRDAVISAAPAVATVGQAALARG
WNPFKELERAGQRVRDAIISAGPAVATVGQAAAIA
WNPFKELERAGQRVRDAIISAAPAVATVGQAAAIARG
WNPFKELERAGQRVRDAVISAAPAVATVGQAAAIARGG
GIGALSAKGALKGLAKGLAZHFAN
GIGASILSAGKSALKGLAKGLAEHFAN
GIGSAILSAGKSALKGLAKGLAEHFAN
IKITTMLAKLGKVLAHV
SKITDILAKLGKVLAIIV
RPDFCLEPPYTGPCKARII
RYFYNAKAGLCQTFVYG
GCRAKRINNFKSAEDCMRTCGGA
FLPLLAGLAANFLPKIFCKITRKC
GIMDTLKNLAKTAGKGALQSLLNKASCKLSGQC
KWKLFKKIEKVGQNIRDGIIKAGPAVAVVGQATQIAK
KWKVFKKIEKMGRNIRNGIVKAGPAIAVLGEAKAL
GWILKKLGKRIERIGQHTRDATIQGLGIAQQAANVAATARG
WNPFKELEKVGQRVRDAVISAGPAVATVAQATALAK
SWLSKTAKKLENSAKKRISEGIAIAIQGGPR
ZFTNVSCTTSKECWSVCQRLHNTSRGKCMNKKCRCYS
FLPLILRKIVTAL
LRDLVCYCRSRGCKGRERMNGTCRKGHLLYTLCCR
LRDLVCYCRTRGCKRRERMNGTCRKGHLMYTLCCR
VVCACRRALCLPRERRAGFCRIRGRIHTPLCCRR
VVCACRRALCLPLERRAGFCRIRGRIHPLCCRR
RRCICTTRTCRFPYRRLGTCIFQNRVYTFCC
RRCICTTRTCRFPYRRLGTCLFQNRVYTFCC
ACYCRIPACIAGERRYGTCIYQGRLWAFCC
CYCRIPACIAGERRYGTCIYQGRLWAFCC
VVCACRRALCLPRERRAGFCRIRGRIHPLCCRR
VVCACRRALCLPLERRAGFCRIRGRIHPLCCRR
VTCYCRRTRCGFRERLSGACGYRGRIYRLCCR
VTCYCRSTRCGFRERLSGACGYRGRIYRLCCR
DFASCHTNGGICLPNRCPGHMIQIGICFRPRVKCCRSW
VRNHVTCRINRGFCVPIRCPGRTRQIGTCFGPRIKCCRSW
NPVSCVRNKGICVPIRCPGSMKQIGTCVGRAVKCCRKK
ATCDLLSGTGINHSACAAHCLLRGNRGGYCNGKAVCVCRN
GFGCPLDQMQCHRHCQTITGRSGGYCSGPLKLTCTCYR
GFGCPLNQGACHRHCRSIRRRGGYCAGFFKQTCTCYRN
ALWKTMLKKLGTMALHAGKAALGAADTISQTQ
GKLPRPYSPRPTSHPRPIRV
GIFSKLGRKKIKNLLISGLKNVGKEVGMDVVRTGIDIAGCKIKGEC
ILPWKWPWWPWRR
FKCRRWQWRMKKLGAPSITCVRRAP
ITSISLCTPGCKTGALMGCNMKTATCHCSIHVSK
TAGPAIRASVKQCQKTLKATRLFTVSCKGKNGCK
MSKFDDFDLDVVKVSKQDSKITPQWKSESLCTPGCVTGALQTCFLQTLTC
NCKISK
KYYGNGVHCTKSGCSVN
WGEAFSAGVHRLANGGNGFW
GIGKFLHSAGKFGKAFVGEIMKS
GIGKFLHSAKKFGKAFVGEIMNS
GMASKAGAIAGKIAKVALKAL
GVLSNVIGYLKKLGTGALNAVLKG
GWASKIGQTLGKIAKVGLKELIQPK
INLKALAALAKKIL
GIGAVLKVLTTGLPALISWIKRKRQQ
ATCDLLSGTGINHSACAAHCLLRGNRGGYCNGKGVCVCRN
ATCDLLSGTGINHSACAAHCLLRGRGGYCNRKGVCVRN
RRWCFRVCYRGFCYRKCR
RRWCFRVCYKGFCYRKCR
RGGRLCYCRRRFCVCVGR
RGGRLCYCRRRFCICV
RGGGLCYCRRRFCVCVGR
VTCDLLSFKGQVNDSACAANCLSLGKAGGHCEKGVCICRKTSFKDLWDKY
F
GWLKKIGKKIERVGQHTRDATIQGLGIAQQAANVAATAR
GWLKKIGKKIERVGQHTRDATIQVIGVAQQAANVAATAR
SDEKASPDKHHRFSLSRYAKLANRLANPKLLETFLSKWIGDRGNRSV
KWCFRVCYRGICYRRCR
RWCFRVCYRGICYRKCR
KSCCKDTLARNCYNTCRFAGGSRPVCAGACRCKIIGPKCPSDYPK
GGKPDLRPCIIPPCHYIPRPKLPR
VKDGYIVDDVNCTYFCGRNAYCNEECTKLKGESGYCQWASPYGNACYCKL
PDHVRTKGPGRCH

Incorporation of a targeting peptide or other targeting moiety into the outer shell may be accomplished by any of the methods known in the art of targeted drug delivery. Suitable methods include but are not limited to covalent attachment of a targeting moiety to one or more components of the outermost shell, either directly or via linkers, binding of biotinylated targeting moieties to avidin or streptavidin molecules attached to the outer shell, and electrostatic binding of appropriately charged molecules, such as the antibodies in the examples below. These and other methods are well known in the art; see for example A. Coombes et al., Biomaterials 18:1153-1161, 1997.

For covalent attachment, chemically reactive groups present on the targeting moiety and on the outer layer of the nanospheres may be coupled to one another by means known in the art. The amino groups provided by the lysine groups of gelatin, for example, can be coupled with activated targeting moieties, such as those where carbodiimides have been used as activating agents for carboxyl groups, rendering them reactive with amino groups. In an alternative embodiment, avidin or streptavidin may be covalently bound to the outer surface of the nanoshells, and biotinylated targeting moieties can then be coupled to the nanoshell surface efficiently. (Wilchek, et al., Meth. Enzmol., 184:5-13, (1990)). Similarly, protein A can be incorporated into the outer shell of the nanospheres and used to bind immunoglobulin targeting moieties.

Shells can be shifted from an “open” state to a “closed” state by changing environmental conditions such as temperature, pH, or by the presence of organic solvents. For shells composed of poly(styrene sulfonate) (PSS) and poly(allylamine hydrochloride) (PAH) multilayers, the penetration of fluorescein is reduced by 3 orders of magnitude upon heating to 80° C. The increased barrier property is believed to be caused by annealing of holes in the shell at the higher temperature. These changes in permeability can be used to load chemical species into the interior of the nanoshells and/or trap them inside the nanoshell.

Even macromolecules such as proteins have been successfully loaded into hollow polyelectrolyte shells, through pH-controlled and water/ethanol mixture-controlled methods. For example, (PSS/PAH)₄₋₅ shells exposed to a pH 4.5 solution form holes of up to 10 nm in diameter, for reasons that are not fully understood. It is possible that changes of the polyelectrolyte charges upon pH variation induce pore formation or loosen the polyelectrolyte network for pore formation. Shells incubated in a 1:1 mixture of water and ethanol are also sufficiently permeable to allow penetration of 5 nm diameter urease proteins through the shell membrane. When these shells are subsequently transferred to water, urease is found in the interior of the shells. Because nanoshell permeability can be controlled by different experimental conditions, bioactive agents may be loaded into the nanoshells of the present invention in several ways.

The bioactive agent may be a diagnostic agent, or a therapeutic agent such as a drug or prodrug. Suitable therapeutic agents include but are not limited to antineoplastic drugs, radiation sensitizers, antibiotics, recombinant or natural proteins, enzyme inhibitors, and receptor agonists and antagonists, and prodrugs thereof. As used herein, the term “prodrug” refers to any substance that is converted in vivo into a different substance which has the desired pharmaceutical activity. In one embodiment, an anticancer drug, such as doxorubicin, is encapsulated in the nanoshells. Drug release kinetics may be controlled by varying the nanoshell membrane properties (e.g., thickness and polymer molecular weights).

The therapeutic agent may also be a radiotherapeutic agent, such as for example a compound or complex of boron or gadolinium, useful in neutron capture therapy, or an inherently radioactive isotope such as ⁵⁵Fe or ¹²⁵I.

The nanoshell membrane in FIG. 1 consists of biocompatible organic polymers and SPIO nanoparticles (˜4 nm), and the nanoshell interior encapsulates a bioactive agent (e.g., doxorubicin). Compared to liposomes, where the membrane consists of lipid bilayers associated through hydrophobic interactions, LbL-assembled nanoshells have a completely different membrane structure consisting of highly charged polymers associated via electrostatic interactions. Consequently, LbL-assembled nanoshells may offer several advantages for drug delivery applications: (1) the membrane thickness may be accurately controlled by the number of polymer layers, which in turn control the membrane permeability and drug release kinetics; (2) a charged nanoshell membrane allows the incorporation of SPIO nanoparticles to generate MRI visibility; and (3) protein modification at the nanoshell surface permits ligand optimization to improve drug targeting efficiency.

Superparamagnetic iron oxide (SPIO) nanoparticles are a class of MRI contrast agents that provide extremely strong enhancement of proton relaxation. In contrast to low molecular weight “T1” paramagnetic metal chelates such as Gd-DTPA, SPIO nanoparticles are classified as T2 negative contrast agents, with MR sensitivity approximately 1000 times higher than T1 agents. SPIO agents are composed of iron oxide nanocrystals which create a large, dipolar magnetic field gradient that creates a relaxation effect on nearby water molecules. According to their sizes and applications, SPIO nanoparticles have been classified into four different categories: large, standard, ultrasmall, and monocrystalline agents. Large SPIO agents are mainly used for gastrointestinal lumen imaging, while standard SPIO agents are used for liver and spleen imaging. When the SPIO nanoparticles are in the range of 20-40 nm (ultrasmall category), they can be injected to visualize lymph node metastases. The smallest monocrystalline SPIO agents are used for tumor-specific imaging when attached to monoclonal antibodies, growth factors, and antigens. In certain embodiments of the present invention, monocrystalline SPIO nanoparticles (diameter ˜4 nm) may be incorporated into nanoshell membranes to introduce MRI contrast.

In certain embodiments, the surface charge of the nanoshells may be modulated. During LbL self-assembly, the outermost layer dominates the surface charge and property. For electrostatic LbL self-assembled shells, the surface charge of the outermost layer is important when interacting with cells. As described herein, 1-micron polyelectrolyte shells with different surface charges and compositions were fabricated and in vitro interactions with the tumor cell line MCF-7 were studied using confocal laser scanning microscopy and flow cytometry. Shell surface charges were characterized by zeta-potential measurements. Polycation coated shells present positive surface charge prior to contacting serum-containing culture media, but surface charge became negative after one hour of culture media incubation. Polyanion coated shells also displayed a surface charge change before and after incubation in serum-containing media. Among all surfaces, shells covered with lipid bilayers displayed the highest cell uptake percentage of 78.7±2.5% after 24 hours shell-cell interaction study. A positive surface charge does not necessarily show a higher cell uptake than a surface with negative charges, and this may due to serum protein adsorption. To prevent protein adsorption, PEI25k-PEG5k copolymers (1:1; 1:5; 1:10) were used as outermost layers for shell assembly. As demonstrated herein, polyelectrolyte shells with a copolymer coating PEI25k-PEG5k (1:10) resulted in the least cell internalization (40.5±0.7% at 24 hours). These in vitro shell-cell interaction results may be useful to tailor the design of shell surfaces for particular applications in drug delivery and molecular sensing.

In certain embodiments, the surfaces of the nanoshells of the present invention may be modified by oligo- or poly-ethyleneglycol regions. This can be done by attaching oligo- or poly-ethylene glycols to the outermost surface of the subject nanoshells, e.g., as pendant side chains, or by including PEG in the outermost polymeric layer of the subject nanoshells, e.g., as a copolymer with a charged polymer, such as a block copolymer.

The effect of particle surface chemistry plays an important role in the uptake process. As described herein, negatively charged proteins were adsorbed on PEI coated shells as indicated by the dramatic change of surface charge. In such case, the amino group on PEI may not play a similar masking function as amidine groups. Compared to negatively charged particles, poly-L-lysine-modified microparticles were internalized under all conditions with highest efficiency, which is suggested to be mediated by their positive surface charge (Thiele L et al (2003) Biomaterials 24(8):1409-18). But in other reports, particles with negatively charged surfaces are easier for cells to uptake compared to positively charged surfaces (Heck J D et al (1983) Cancer Res 43:5652-6). In general, the neutral surface is considered the surface with the least cell uptake than positive or negative surfaces. For example, as described herein, PEG coated particles such as particles coated with PEI25k-PEG5k (1:10) have a much lower cell uptake compared to shells with other materials.

Shell surface lipophilicity may also play an important role in cell uptake process. Lipid bilayers on shell surfaces presented the highest the negative charges and resulted in the highest particle uptake. Similar results were also reported in other studies such as lipoprotein uptake, and it was suggested that the presence of lipoprotein lipase was very helpful to facilitate the uptake of lipoproteins (Rinninger, F et al (1998) J Lipid Res 39(7):1335:48). As described herein, 1-micron shells are much bigger than lipoplex (Ross, P C et al (1999) Gene Ther 6(4):651-9) and lipoprotein based drug delivery systems. The mechanism behind this high percentage of shell uptake is unknown but may be due to the lipophilic property that helps cell membrane fusion with lipid shells. The ability to incorporate lipid bilayers into polyelectrolyte shells is noteworthy since it represents a unique system with combined physical and chemical properties. The special structure leads to function changes of which the shell membrane permeability is greatly reduced (Moya, S et al (2000) Macromolecules 33:4538-4544). It is important to combine the shell surface physical properties with other characteristics such as surface charge to determine how cells will respond to these microparticles.

It is known that the presence of albumin on particle surface usually decreases uptake by cells (Moghimi, S M et al (1993) Biochim Biophys Acta 1179:157-65; Thiele, L (2003) Biomaterials 24(8):1409-18). This may be due to reduced opsonization of shells due to the dysopsonic activity of albumin. But different cells may react differently to albumin-coated particles. For example, uptake experiments conducted with respiratory epithelium cells indicated that albumin-coated microspheres were neither bound nor internalized by the Calu-3 cells but internalized by A549 cells as large as 3 micron (Foster, K A et al (2001) 53(1):57-66).

For the positively charged polymer shells, no direct relationship is presented between surface charge and particle uptake. Both PDDA and PEI25k-PEG5k (1:1) layers have the highest shell uptake percentages among all positive charge formulations. PEI25k-PEG5k (1:10) has the lowest shell uptake, which is may be due to the shielding property of grafted PEG. PLL coated particles present the second lowest internalization of 47.4±2.7% after 24 hours incubation. While high phagocytotic activities of macrophages and dendritic cells were observed in another study when microparticles (1 microns) were coated with PLL instead of albumin (Thiele, L et al. (2001) J Control Release 76(1-2):59-71). Thiele et al. suggested that phagocytotic activity of those cells largely depends on particle size and surface charge and is also influenced by the character of bulk and coating material. As described herein, shells covered with PLL or albumin display similar uptake ratios of 47.4% and 47.5% without statistical difference (P>0.05). This may be because MCF-7 cells have different phagocytotic activity compared to macrophages and dendritic cells.

As described herein, to ensure that shell-cell interactions are dependent on shell surface charge and other properties but not due to the cytotoxicity caused by shells, a three-day shell cytotoxicity study was conducted. None of the shell formulations showed cell cytotoxicity in vitro as compared to the negative controls. To avoid the effects due to serum protein adsorption on shell surfaces, a parallel study was also carried out in order to observe shell behavior in a serum-free media. At the beginning of the study, original culture media was replaced with HBSS solution containing shells. A short time (one hour) incubation was performed as to prevent any side effects that may occur due to the absence of serum. Still, most cells (about 80%) were detached and the flow cytometry data showed that all cells internalized shells and that there was no difference between different formulations. In this situation, the flow cytometry data from in serum-containing media was analyzed.

As described herein, polyelectrolyte shells with different outermost layers were assembled and the shell-cell interactions were studied in vitro. Negatively charged lipid bilayers presented the highest shell uptake ratio in MCF-7 cells while copolymer PEI25k-PEG5k (1:10) displayed the lowest uptake ratio. A positive surface charge does not necessarily equal higher shell internalization. For shells with three different negative charges, higher cell internalization corresponds to a higher surface charge. Both PDDA and PEI25k-PEG5k (1:1) layers have the highest shell uptake percentages among all positive charge formulations. Shell original surface charges were dramatically changed after one-hour incubation in serum-containing media. Proteins such as albumin and globulin may be adsorbed onto polycations while other positively charged materials may interact with polyanions. The shielding effect of PEG was noticed since less surface charge was related to a higher PEG grafting ratio.

The simplicity and versatility of LbL self-assembly provides a unique method to build hollow polyelectrolyte shells that may be used as drug carriers or fluorescence sensors. The data described herein may be useful when designing shell surface properties for different biomedical applications such as in cancer therapy.

By modulating the surface charge of the nanoshells of the subject invention, the amount of nanoshells of the subject invention taken up by cells (e.g., breast cancer cells) may be increased or decreased. In certain embodiments, the surfaces of the nanoshells of the subject invention are modified by PEG. In certain further embodiments of the invention, PEG-modified shell surfaces reduce protein adsorption and may provide prolonged blood circulation for drug delivery applications.

### EXAMPLE I

In drug delivery applications, smaller particle diameter (<1 μm) is important for prolonged blood circulation and enhanced drug targeting to specific body sites. The size of a self-assembled polymer shell directly correlates with the core size. In this example, monodisperse, decomposable MF particles ranging from 1 to 5 μm in diameter, obtained from Microparticles GmbH (Berlin, Germany), were the source of the cores.

In order to further reduce the particle size, the particles were subjected to a surface-erosion procedure. It has been reported that complete MF particle decomposition occurs after 20 seconds in a pH 1.1 HCl solution (C. Gao et al., Macromol. Mater. Eng. 286 (2001) 355). Applicants have discovered that at higher pH values (>1.9), MF particle size is gradually reduced through surface degradation. By way of example, monodisperse 1.2 μm MF particles (Microparticles GmbH, Germany) were suspended in acidic HCl solutions (6×10⁵ particles/ml) with a specific pH value from 1.9 to 2.2. At different times, particle suspensions were analyzed by dynamic light scattering (DLS) (90Plus Submicron Particle Size Analyzer, Brookhaven Instruments) to measure the particle diameters in solution at room temperature. Particle surface charge before and after treatment was also characterized by zeta-potential measurement in 1 mM KCl solution.

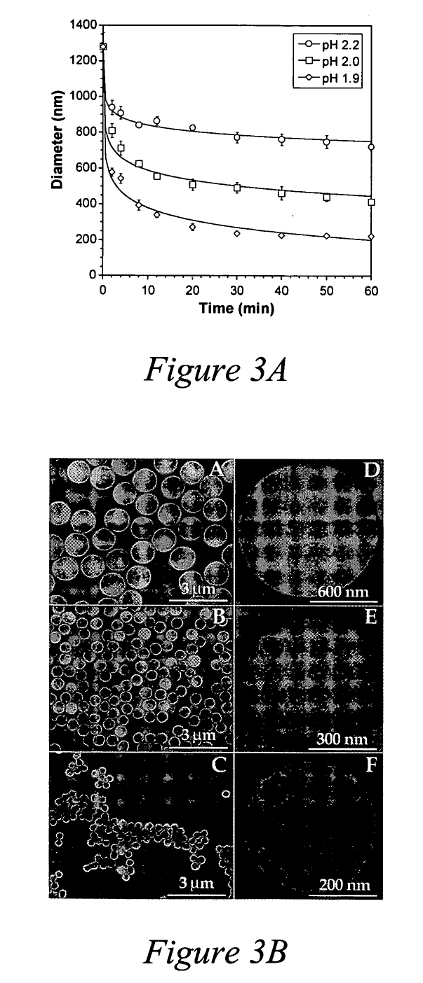

FIG. 3A shows the particles' diameter (as measured by DLS) as a function of decomposition time at pH values 1.9, 2.0, and 2.2. The original MF particle size was determined to be 1279±79 nm. After 60 minutes, a particle diameter of 222±11 nm was obtained in the pH 1.9 suspension. Treatment with pH 2.0 and pH 2.2 HCl solutions for 60 minutes led to particle diameters of 415±30 and 702±11 nm, respectively. Decomposition kinetics at these pH values clearly deviates from the previous observation of linear size reduction over time at pH 1.1 (Gao et al., Macromol. Mater. Eng. 286 (2001) 355). At the higher pH values employed in the present invention, a dramatic particle size decrease in the first 20 minutes was observed in all of the HCl solutions. More specifically, particle size decreased from 1279±79 nm to 826±18 nm, 509±31 nm, and 273±19 nm after 20 min hydrolysis in pH 2.2, 2.0, and 1.9 solutions, respectively. These sizes correspond to 69%, 42%, and 23% of the original particle diameter. Between 20 and 60 minutes, the particle size decreased more slowly with less than 20% size reduction. Logarithmic curve fitting (KaleidaGraph™ data analysis and graphing software, version 3.06) provided a reasonable fit to the experimental decomposition kinetics (FIG. 3A). The data in FIG. 3A demonstrate that particle diameter depends on both hydrolysis time and pH values. Prolonged acid treatment (>20 minutes) at precisely-controlled pH values is a preferred embodiment for the reproducible control of particle sizes according to the present invention. The acid-hydrolyzed, size-reduced MF particles are positively charged and have similar zeta-potential to the original larger particles. For example, the zeta-potential for the 373 nm MF particles is 28.7±6.3 mV (n=3), whereas that for the unmodified MF particles 29.4±3.5 mV (n=3).

Scanning electron microscopy (SEM, Hitachi S-4500 model) was used to further characterize the particle size and surface morphology. FIG. 3B shows the SEM images of MF particles before hydrolysis, and after 8 and 60 minutes hydrolysis in pH 2.0 HCl solution. The SEM image of the original MF particles confirms the monodisperse distribution of these particles (Panel A). The particle diameter from SEM analysis is 1183±25 nm, which is slightly smaller than the DLS measurement (1279±79 nm). At higher magnification (×25K), the original MF particles are shown to be spherical, with a smooth surface (Panel D). Panels B and C demonstrate that the MF particles surprisingly maintained their spherical shape and monodisperse size distribution after 8 and 60 minute hydrolysis, respectively. Although rapid diffusion of acid into sub-micron particles of hydrated polymer might be expected, the relatively smooth surface morphology of the acid-hydrolyzed MF particles (Panels E and F) suggests that a surface-erosion process is occurring under these conditions.

Based on the SEM images, the particle diameters were 597±15 and 373±18 nm for MF particles after 8 and 60 minute hydrolysis, respectively. These values are lower than those measured by dynamic light scattering (650±20 and 430±30 nm after 8 and 60 minute hydrolysis, respectively), most likely reflecting particle shrinkage due to dehydration during sample preparation for SEM analysis. Using the LbL self-assembly technique, a series of sub-nanometer polymer nanoshells with different diameters were fabricated, using the different sizes of MF particles prepared above as templates. Capsules composed of gelatin multilayers are known to effectively extend drug release half-life (H. Ai, S. Jones, M. De Villiers, Y. Lvov, J. Control. Release 86 (2003) 59), and nanoshells comprising layers of gelatin represent a preferred embodiment of the present invention. By way of example, MF particles were first suspended in 2 mg/ml gelatin solution in pH 7.4 PBS for 30 minutes. Centrifugation was used to remove excess polymer before coating the next layer. A second layer of poly(dimethyldiallyl ammonium chloride) (PDDA, MW=200 kD, Polysciences) was applied following a similar coating procedure. The process was repeated, until a total of five bilayers of gelatin and PDDA were introduced to the MF particle surface. Subsequently, core decomposition was carried out in a pH 1.2 HCl solution for 2 minutes, followed by removal of degraded MF oligomers. Hollow polyelectrolyte nanocapsules composed of (gelatin/PDDA)₅ were thus obtained. It should be noted that choosing appropriate pH during assembly is important to maintain a sufficiently high degree of ionization for the charged polymers. Gelatin is a protein-based zwitterionic molecule with an isoelectric point of 4.5, and sufficiently high pH values are necessary to maintain the polyanionic state of the molecule for LbL self-assembly.

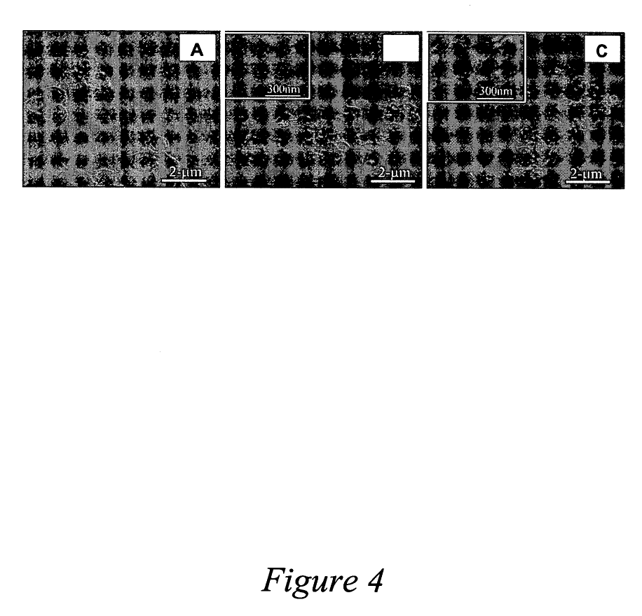

FIG. 4 shows SEM images of three sets of nanoshells produced with MF particles of 1.2 μm, 590 nm and 360 nm in diameter. These nanoshells consisted of five alternating bilayers of gelatin and poly(dimethyldiallyl ammonium chloride) (PDDA). Due to the fact that the inner MF cores were completely dissolved away, SEM images showed collapsed shell structure as a result of sample drying process. Collapsed shells (600 and 390 nm) appeared to be slightly larger than the diameters of the MF cores (590 and 360 nm). Magnified inserts in FIGS. 4B and 4C further show the morphology of individual nanoshells.

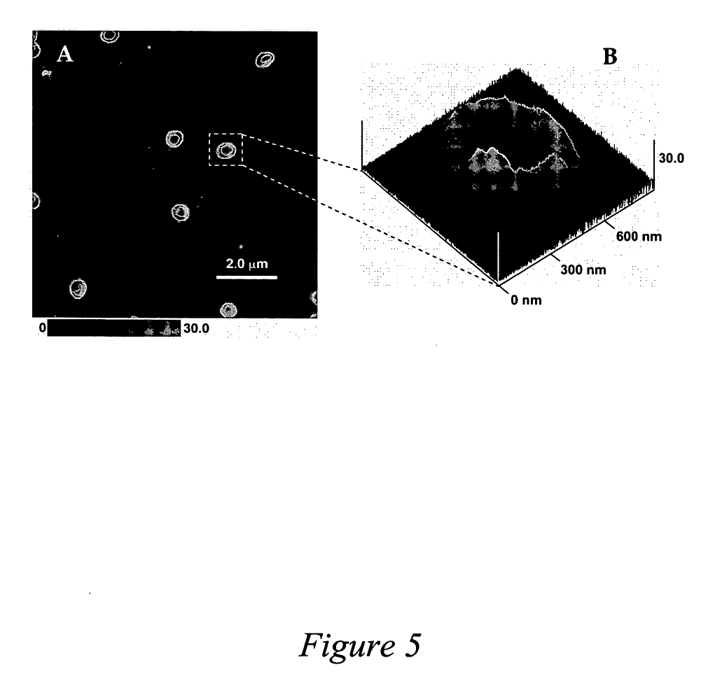

Polymer nanoshells were further characterized by scanning force microscopy (SFM) using a Nanoscope III Multimode SFM (Digital Instrument Inc., Santa Barbara, Calif.). Samples were prepared by applying a drop of the nanoshell solution onto a freshly prepared mica substrate. Since mica is slightly negatively charged and nanoshells have a positively charged PDDA outermost layer, electrostatic interactions are sufficient to anchor the nanoshells on the mica surface. The sample was extensively washed with Millipore deionized water and dried under a gentle stream of nitrogen. SFM images were recorded in air at room temperature with tapping mode measurement. FIG. 5A shows the SFM images of multiple 620 nm nanoshells. Similar to the SEM data, SFM images showed a uniform distribution of polymer nanoshells with collapsed shell morphology. The diameter of these shells from SFM analysis (˜620 nm) is consistent with those from SEM analysis. FIG. 5B shows an individual nanoshell in 3-dimensions. The nanoshell is in ring shape due to the polymer folds at the shell boundary. SFM allows the measurement of the height of the folding as approximately 30 nm.

It is known that polymer shells can shift from an “open” state to a “closed” state by changes in environmental conditions such as pH, or in the presence of organic solvents. Shells composed of (gelatin/PDDA)₅ multilayers (5 μm in diameter) were loaded with doxorubicin (DOX) by putting the. shells in an “open” state by lowering the pH to 3, and incubating the shells in DOX solution (2 mg/ml).

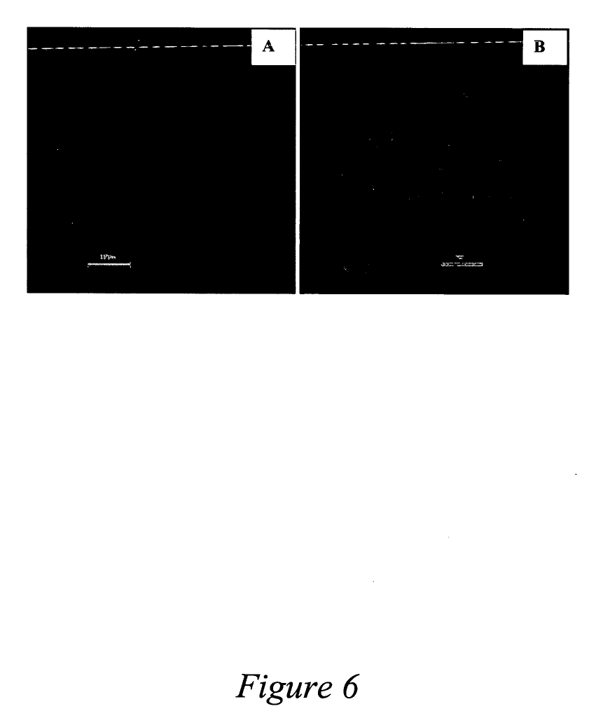

FIG. 6A shows the fluorescence confocal microscopy image of polymer shells in DOX solution at pH 3. It should be noted that DOX is a fluorescent drug (λₑₓ=479 nm, λₑₘ=593 nm). At this pH, the shell membrane was highly permeable to DOX and within 10 minutes, DOX reached the same concentration inside the shells as in solution. After DOX loading, the shells were washed with PBS buffer (pH 7.4) to remove the free DOX molecules in solution and “close” the shells. FIG. 6B demonstrates the successful loading of DOX into the shells. In these confocal images, DOX was found only inside the shells and within the shell membrane. The affinity of DOX to the shell membranes is most likely due to interactions between the positively charged ammonium groups on the DOX molecule with the polyelectrolyte membrane.

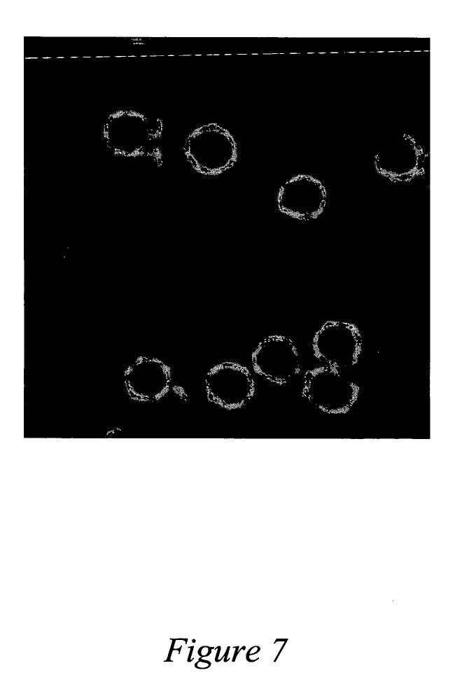

The feasibility of constructing polymer shells from biocompatible organic polymers was demonstrated with positively charged poly-L-lysine (PLL, MW=70 kD) and negatively charged gelatin (MW=40 kD). The shells were composed of five bilayers of gelatin and PLL, fabricated using the usual LbL self-assembly procedure. After shell fabrication, the shell surface was coated with a labeled and negatively-charged protein, albumin-FITC (PI=4.7). FIG. 7 is a confocal microscopy image of the albumin-attached shells, clearly showing the fluorescence layer of albumin on the (gelatin/PLL)₅ shells. To applicants' knowledge, this represents the first example of a polymer nanoshell completely composed of biocompatible and biodegradable organic polymers. The ability to assemble proteins such as albumin at the shell surface demonstrates the feasibility of coating such nanoshells with antibodies, such as anti-Her2/neu monoclonal antibodies, for targeting purposes.

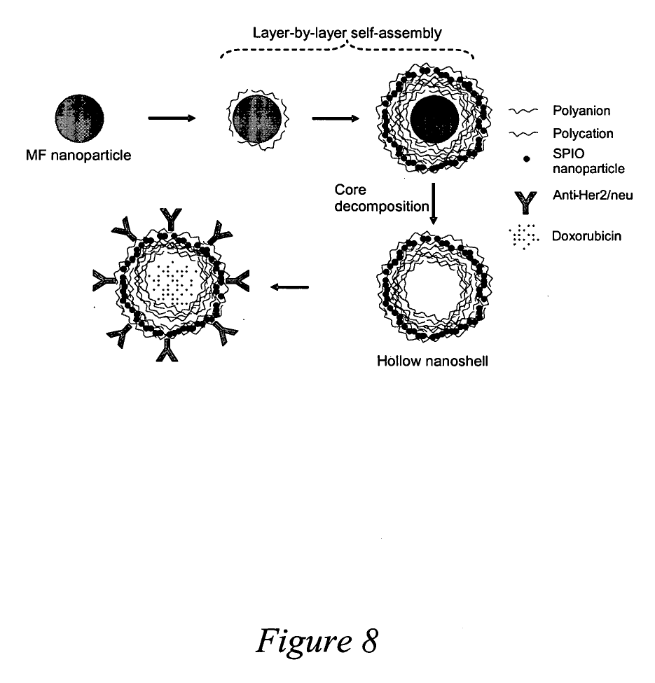

FIG. 8 illustrates the overall fabrication procedure for preparing one embodiment of the invention. First, weakly crosslinked MF nanoparticles are incubated in a polyanion solution for 30 minutes to allow saturation adsorption of polyions on the colloidal surfaces. Excess polymers are then removed before the next layer of coating. Magnetic nanoparticles are incorporated into the shell structure at this stage. After LbL self-assembly, MF core decomposition will be carried out in a 0.1 M HCl solution. Finally, doxorubicin is loaded into the shells and anti-Her2/neu is coated on the outermost layer for targeting purposes.

To control the nanoshell size, a series of MF particles with diameters at 100, 300, and 600 nm were first obtained. To precisely control the MF particle size, a working curves (such as FIG. 3) correlating particle size with treatment time in HCl solutions of different pH values is consulted. Both treatment time and pH value may be used to control the final MF particle size. Anionic biopolymer gelatin is applied as the first layer to coat the positively charged MF particles. Generally, 2 mg/ml gelatin in PBS buffer (pH 7.4) is used. The incubation time was approximately 30 minutes to ensure full polymer coverage. Ultracentrifugation removes excess polymers before coating the next layer. In total, five bilayers of gelatin and PLL are coated on MF particles to form the shells. Subsequently, core decomposition will be carried out in a pH 1 HCl solution for 5 minutes. Extensive wash of shells in pH 1 HCl solution through centrifugation/redispersion procedure is essential to remove MF oligomers. Hollow polymer nanoshells will be obtained and dispersed in PBS for storage.

To monitor the step-by-step coating process, growth of each polymer layer is monitored by measuring the electrostatic potential of coated particles, e.g., with a Brookhaven Zeta Potential Analyzer. A layer of PLL coating will lead to a positive zeta-potential while a negative zeta-potential represents a layer of gelatin coverage. Alternating positive and negative zeta-potentials indicates a successful layer-by-layer self-assembly process.

Scanning force microscopy (SFM) allows for high-resolution investigations on a sub-micrometer level and has been used in shell surface morphology study. Compared to SEM, SFM can show more quantitative details about the shell structure such as sample height (see FIG. 5). Moreover, SFM is particularly helpful in investigating 100 nm shells (this size reaches spatial resolution limit for SEM to characterize organic materials). Shell samples are prepared on mica through an established method. SFM images using tapping mode are recorded in air at room temperature using a Nanoscope III Multimode SFM (Digital Instrument Inc., Santa Barbara, Calif.).

Nanoshells are incubated in 2 mg/ml DOX solution (0.9% NaCl) at pH 3. Once concentration equilibrium is reached across the shell membrane (10 min), PBS buffer (pH 7.4) is used to remove free drug molecules and “close” the shells. This procedure was successfully carried out in 5 μm shells (FIG. 6), and the loading parameters established with 5 μm particles are used, with a fluorescence spectrophotometer (e.g., LS-45 model, Perkin-Elmer) being used to quantify the loading density of DOX inside the nanoshells. Release studies are carried out at 37° C. in PBS buffer (pH 7.4). The released DOX is separated from the nanoshell-encapsulated DOX and quantified by HPLC (Series 200 pump, Perkin-Elmer; C18-reverse phase column, pH 7.0 ammonium acetate buffer).

By the above method, nanoshells having 5, 8, and 12 polymer bilayers are prepared, using various polymer molecular weights (gelatin: 20, 40, and 80 kD; PLL: 10, 30, and 100 kD) in the shell membrane.

Charged and monodisperse superparamagnetic iron oxide (SPIO) nanoparticles of ˜4 nm in diameter are incorporated into the nanoshell membranes. Optimization of the MRI acquisition conditions to maximize the sensitivity of NMR detection is within the ability of those skilled in the art. Under these optimized conditions, the relaxation rates (1/T1, 1/T2, and 1/T2*) of the shells is determined, and calibration curves between MR intensities and nanoshell concentrations, for nanoshells with different diameters and densities of SPIO nanoparticles, are established.

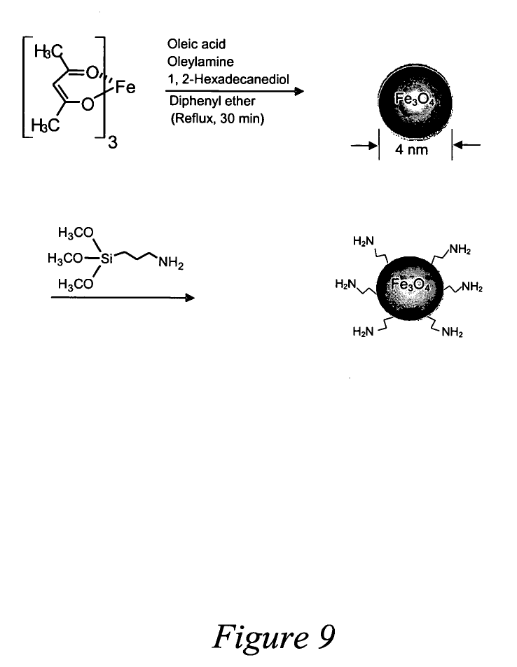

Using monodisperse ultrasmall SPIO nanoparticles is important in shell assembly. This ensures homogeneous magnetic property for each shell. An organic-phase synthesis of magnetite (Fe₃O₄) nanoparticles with uniform size distribution is employed, by which average size can be controlled from 3 to 20 nm in diameter. The 4 nm Fe₃O₄ nanoparticles are synthesized following this procedure (FIG. 9, step 1). Iron(III) acetylacetonate (Fe(acac)₃, 2 mmol) is mixed with 1,2-hexadecanediol (10 mmol), oleic acid (6 mmol), and oleylamine (6 mmol) in diphenyl ether (20 ml) under nitrogen and heated to reflux at 265° C. for 30 min. After cooling to room temperature, the solution is treated with ethanol under air, and a dark-brown material precipitates from the solution. The supernatant is removed through centrifugation, the pellets are dissolved in hexane in the presence of oleic acid and oleylamine, and then reprecipitated with ethanol to give monodisperse 4 nm Fe₃O₄ nanoparticles.

The Fe₃O₄ nanoparticles are well-dispersed in hexane but not in water. Surface modification of these nanoparticles is necessary to introduce particle surface charge for aqueous dispersity and shell incorporation. Silanization is known to be an efficient method for modifying Fe₃O₄ nanoparticle surface properties, and NH₂-terminated trimethoxysilane is accordingly used for particle surface modification (FIG. 9, step 2). The surface charge and zeta-potential of the resulting nanoparticles are determined by measuring the microelectrophoretic mobility of the particles. Positively-charged Fe₃O₄ nanoparticles are expected at neutral pH.

A similar LbL assembly procedure is used to produce nanoshells impregnated with SPIO nanoparticles. The initial polyelectrolyte multilayer film is (gelatin/PLL)₄+gelatin. This film provides a uniformly negatively charged surface and facilitates subsequent adsorption of positively charged SPIO nanoparticles. Electrostatic interactions between the cationic SPIO nanoparticles and anionic gelatin are the driving force to build the nanocomposite multilayers.

By the above method, nanospheres having one to four bilayers of (Fe₃O₄/gelatin) incorporated in the shell membrane are produced. MF core dissolution and nanoshell purification are done as described above. The final nanoshells are characterized by dynamic light scattering, scanning force microscopy and transmission electron microscopy.

For SPIO contrast agents, MR signal intensity depends on various factors, including particle size, composition, concentration, and data acquisition parameters. Equation 1 describes the signal expression for a spin-echo acquisition, where TR and TE are spin-echo data acquisition parameters and ρ is spin density. Equation 2 shows the relationship for a spoiled gradient echo acquisition.
S(TR,TE)≅ρe^{-TE/T2}(1−e^{-TR/T1}) (1)

$$S ( TR , TE ) ≅ \frac{ρ sin ( α ) ( 1 - {ⅇ}^{- TR / T1} ) {ⅇ}^{- TE / T2 *}}{1 - cos ( α ) {ⅇ}^{- TR / T1}} \qquad ( 2 )$$

Agents that shorten T1 cause higher signal intensity, since the term (1−e^{-TR/T1}) increases as T1 decreases (at fixed TR). Agents that shorten T2 or T2* cause reduced signal intensity, since the term e^{-TE/T2} decreases as T2, T2* decrease (at fixed TE). SPIO contrast agents have dramatic effects on T2 and T2* shortening which leads to signal intensity reduction. T1 and T2 relaxation rates are usually expressed as the inverse (1/T1, 1/T2, unit: sec⁻¹) and plotted against the concentration of Fe₃O₄. The slopes of these two curves correspond to R1 and R2 relaxivity, respectively. Usually, over ranges used in-vivo, a linear relationship exists between the concentration of contrast agent and the relaxation enhancement. Higher concentration of the Fe₃O₄ led to higher relaxation rates (sec⁻¹).

For in vitro MR studies, agar gel tissue phantoms (1%) with SPIO nanoshells are prepared. These are agar gels with different shell concentrations at 0, 10⁹, 2×10⁹, 4×10⁹, and 8×10⁹ shells/ml. The shell concentrations in the phantoms correspond to Fe₃O₄ concentrations of 0, 0.1, 0.2, 0.4, and 0.8 mM (assuming one layer of SPIO nanoparticles in 300 nm diameter shells), respectively. At the concentration of 10¹⁰ shells/ml, 600, 300, and 100 nm shells are prepared in different agar gels. For 300 nm shells, three groups containing 1, 2, 3 and 4 bilayers of (Fe₃O₄/gelatin) are be used to study the design of layered structure of SPIO nanoparticles. The variable parameters are summarized in Table 2.

TABLE 2
Nanoshell concentration, size, and composition in MR studies

| Parameter 1^{a} | Parameter 2^{b} | Parameter 3^{c} |
|---|---|---|
| Shell Concentration | Shell Size | Shell Composition |
| 3 × 10⁹ shells/ml | 100 nm | (PLL/gelatin)₄ + |
| | | Fe₃O₄/gelatin)₁ |
| 6 × 10⁹ shells/ml | 300 nm | (PLL/gelatin)₄ + |
| | | (Fe₃O₄/gelatin)₂ |
| 1.2 × 10¹⁰ shells/ml | 600 nm | (PLL/gelatin)₄ + |
| | | (Fe₃O₄/gelatin)₃ |
| 2.4 × 10¹⁰ shells/ml | 900 nm | (PLL/gelatin)₄ + |
| | | (Fe₃O₄/gelatin)₄ |

^{a}Shell composition: (PLL/gelatin)₄ + (Fe₃O₄/gelatin); shell size: 300 nm.
^{b}Shell composition: (PLL/gelatin)₄ + (Fe₃O₄/gelatin); shell concentration: 6 × 10⁹ shells/ml.
^{c}Shell size: 300 nm; shell concentration: 6 × 10⁹ shells/ml.

MR studies are carried out on a 1.5 T Siemens Vision MR Imager (Erlangen, Germany) and a 7T Biospec System (Bruker BioSpin Corporation, Billerica, Mass.). T1 and T2 relaxation times are measured in milliseconds at 37° C. Before each measurement, the spectrometer/imager is tuned to the proton resonance frequency and the RF pulses are calibrated. For phantom T2 calculation, multi-echo spin-echo (SE) images (TR/TE=4000 msec/20, 40, 60, and 80 msec; one excitation) are obtained. For phantom T1 calculation, SE images with variable TR (TR=4000, 1000, 500, 200, and 100 msec) and 8 msec TE are obtained with the same receiver gain. For phantom T2* calculation, T2*-weighted Fast Low Angle Shot acquisitions with RF spoiling (FLASH; TR/TE/flip angle=120 msec/4, 8, 12, and 16 msec/30°; one excitation) are obtained separately at each TE with the same receiver gain. This sequence is known as a GRASS acquisition on other commonly available clinical systems. While there are a myriad of other MR methods that have been proposed to estimate T1, T2 and T2*; these have been selected for simplicity and relative accuracy. Other established methodologies may also be employed (e.g., CPMG pulse sequences, Inversion recovery, etc.) to determine and verify the values of T1, T2 and T2*.

Signal amplitude (SA) is determined by using homogeneous region of interest (ROI) (>100 voxels) measurements from each phantom. T1, T2 and T2* are calculated using simple logarithmic relationships for T2 and T2* and by exponential curve fitting to the signal amplitude curves versus TR for estimates of T1. 1/T1, 1/T2 and 1/T2* are plotted as a function of iron concentration. In all experiments, the correlation coefficient r of the fit curve is used to test the linear relationship between relaxation rates and concentration of Fe. Values of R1, R2* and R2 for the different shell designs are compared in order to select preferred embodiments.

To target nanoshells to breast cancer cells in vitro, anti-Her2/neu monoclonal antibodies are attached to the nanoshell surface. The cell targeting efficiency is monitored by MRI and further correlated to results from flow cytometry.

Anti-Her2/neu monoclonal antibodies conjugated with FITC (Becton Dickinson Immunocytometry Systems) are coated as the outermost layer on the hollow nanoshells for targeting purposes. The density of antibody adsorption on the nanoshells may be indirectly estimated by coating antibodies on quartz crystal microbalance electrode. AU-565 breast cancer cell line is preferred, due to the high Her2/neu expression level. Two other cell lines, MCF-7 and MDA-MB-231 cells, with low expression level of Her2/neu, are used as controls. Targeting of nanoshells to the cells is analyzed in vitro. Different concentrations of anti-Her2/neu-coated nanoshells are incubated in the cell culture media (protein free) for 30 minutes. Albumin-FITC-coated polymer nanoshells are used as a negative control. Cells are harvested from the flasks using enzyme-free cell dissociating buffer (Invitrogen, Carlsbad, Calif.) at room temperature followed by extensive washing to remove excess nanoshells in solution. For each sample, 10⁶ cells are used in the flow cytometry study. All data are acquired with a FACScan flow cytometer (Becton Dickinson, San Diego, Calif.). Acquisition parameters are optimized for detection of FITC fluorophore. Ten thousand events are counted for cells with different shell targeting. Expression levels of Her2/neu are quantified using fluorescence of standard calibrated microspheres. The percentage of shell targeting is determined from the calibration curve. An MTT assay is used to determine the cytotoxicity of DOX-containing nanoshells to AU-565 cells.

Cell targeting efficiency of SPIO-incorporated nanoshells is investigated by MR imaging using optimized parameters established as described above. After cell uptake of different amount of nanoshells, cells are washed to remove any unbound SPIO-incorporated nanoshells. Then the cells are detached from the flask and fixed with 2% paraformaldehyde in PBS, and 10⁷ cells are embedded in 1% agar gel (1 ml). The necessary concentration of cells/ml for MR image is determined from the cell targeting efficiency results obtained above. A reference sample containing different concentrations of magnetic nanoshells (0, 3×10⁹, 6×10⁹, 1.2×10¹⁰, and 2.4×10¹⁰ shells/ml) is similarly prepared. Under optimized acquisition conditions as established above, the MR intensity of cancer cells with different amount of SPIO nanoshells inside is measured. The MR intensity is correlated to the nanoshell uptake as quantified by flow cytometry (Section D.3.1). Finally, the values of R1 and R2 of cancer cells containing SPIO nanoparticles are determined, and compared to those from agar gel tissue phantoms.

### EXAMPLE II

Cationic poly(dimethyldiallyl ammonium chloride) (PDDA, MW 200 KD, Aldrich), and negatively charged polypeptide, gelatin (Sigma) were selected for the LbL assembly. Solutions of 2 mg/mL PDDA, and 3 mg/mL gelatin were prepared in phosphate buffered saline (PBS). 1,2-Dipalmitoyl-sn-Glycero-3-Phosphocholine (DPPC), 1,2-Dipalmitoyl-sn-Glycero-3-Phosphate (DPPA), and NBD labeled DPPC were obtained from Avanti Polar Lipids, Inc. 2 mg/mL Doxorubicin (DOX) HCl (Bedford Laboratories™, Bedford, Ohio) was preserved in a 0.9% sodium chloride pH 3 solution. 50-nm silica particles were obtained from Polysciences, Inc. Weakly crosslinked Melamine formaldehyde (MF) particles, 5 micron in diameter, was obtained from Microparticles GmbH, Germany. Human breast cancer MCF-7 cells were obtained from ATCC.

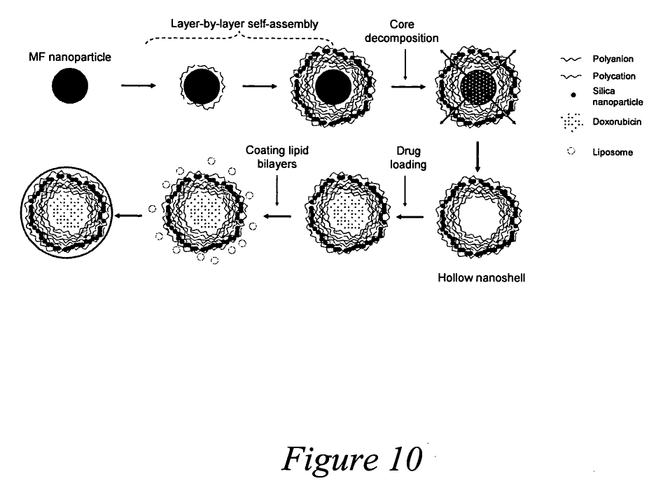

The shell fabrication procedure is illustrated in FIG. 10. MF microparticles were used as templates and incubated in gelatin solution. 50 minutes of coating was performed before washing away free polymers. A second layer of PDDA was added on by using the same coating procedure of the first layer. Five bilayers of gelatin/PDDA were assembled. Then another bilayer of SiO2/PDDA was further coated. The coated particles were exposed to pH 1 HCl solution for core decomposition. Hollow shells were obtained after washing. 100 nm liposomes (85% DPPC, 10% DPPA, 5% NBD-DPPC) were fabricated through mechanical extrusion. Doxorubicin loading into shells was carried out at pH 3 for 30 minutes. Washing with PBS was followed to remove free DOX in solution. Final lipid bilayers were assembled on hollow polymer shells through an established protocol (Moya, S et al (2000) Macromolecules 33:4538). Release kinetics was studied in pH 7.4 PBS at 37° C. Free DOX solution, DOX-loaded shells, and empty shells of different concentrations were used in cell cytotoxicity studies for 5 days.

Fabrication of nano-organized shells:

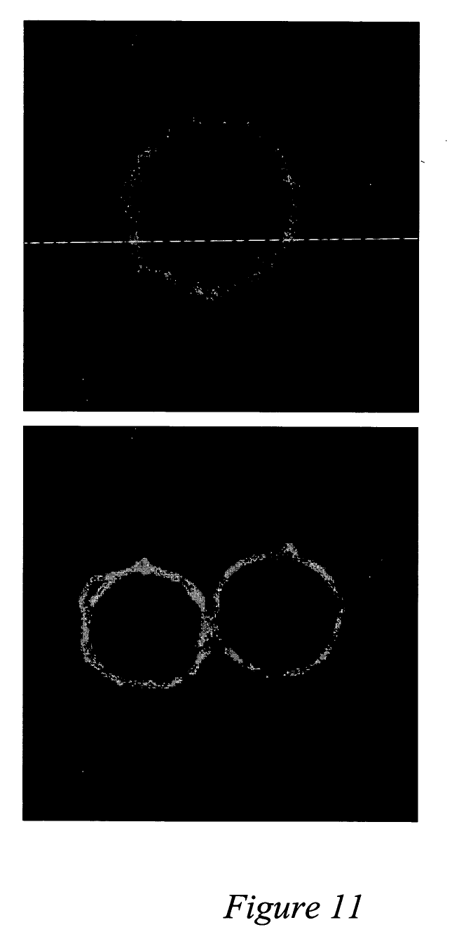

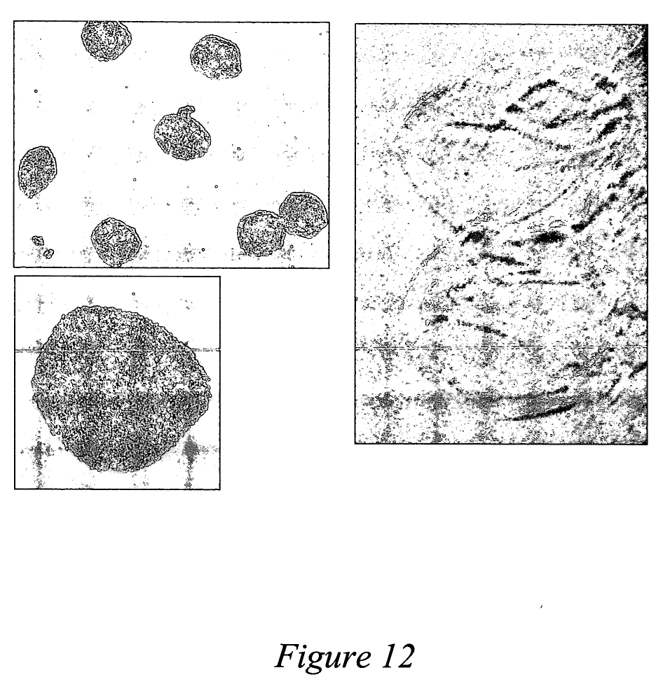

Microshells were fabricated through the LbL self-assembly process and studied under SEM (FIG. 11). In FIG. 11A, 5 μm shells composed of (gelatin/PDDA)₅, silica, and PDDA were monodispersed in size. No obvious aggregation was found by searching different areas under SEM at lower magnification. One shell (surrounded with a dashed box) in FIG. 11A was viewed at a higher magnification in FIG. 11B. Clearly, 50 nm silica nanoparticles were covered on the shell surface. In FIG. 11C, two shells composed of (gelatin/PDDA)₅ were also viewed under SEM. All shells were flat and this may due to the drying process during sample preparation. Shells coated with lipid bilayers were studied under confocal microscope.

In vitro release of doxorubicin:

Approximately 36 μg doxorubicin was encapsulated per 10⁹ shells. Shells were incubated in free doxorubicin solution for 30 minutes and washed through centrifugation to remove the unencapsulated drug. Final shell pellets were dispersed in PBS solution at pH 7.4. The in vitro release was studied at 37° C. in pH 7.4 PBS solution. About 50% drug was released at 8 hours. FIG. 6 shows loading of doxorubicin into polymer shells composed of (gelatin/PDDA)₅, silica, and PDDA. Panel A depicts co-incubation of free drug doxorubicin with shells at pH 3-4. Panel B shows shells loaded with doxorubicin in PBS at pH 7.4.

Cytotoxicity study:

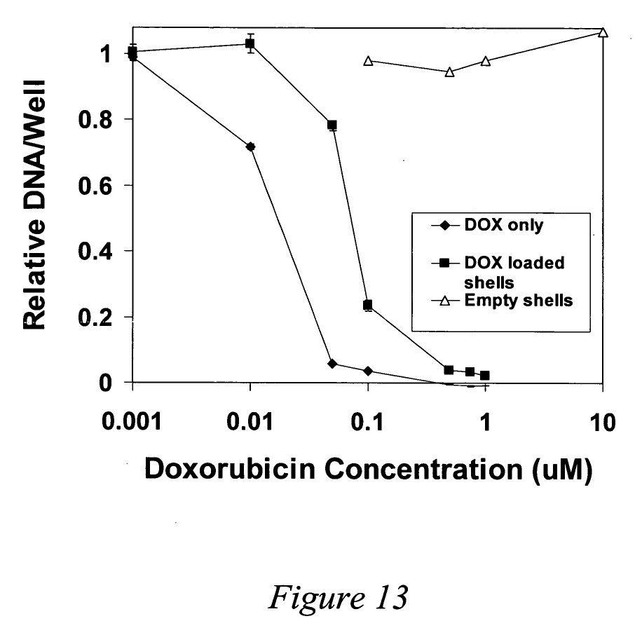

A cytotoxicity study of DOX loaded shells was carried out in vitro as compared to free doxorubicin solution (FIG. 13). DOX loaded shells have a statistically higher IC50 than free DOX solution (p<0.01). Empty shells did not show cytotoxic effects.

FIG. 13 shows the cytotoxicity study of doxorubicin loaded shells compared to doxorubicin solution and empty shells. All samples were incubated in culture media for 5 days. Doxorubicin solution has an IC50 at 0.012±0.002 μM, and doxorubicin loaded shells have an IC50 of 0.075±0.005 μM. There is a statistical difference (p<0.01) between two IC50s. Empty shells did not show obvious cytotoxic effects on MCF-7 cells.

Shell-cell interaction/uptake study:

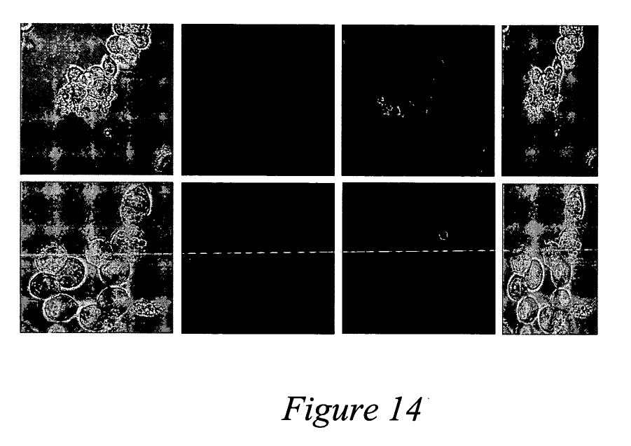

Shells (5 mm in diameter) without drug loading were incubated in culture media for 1.5, 5, and 30 hours in order to study the shell-cell interactions. At different time points, almost all shells were outside of cells and attached to the cell membrane (FIGS. 14E, F, G, and H). It was only found in one case that a shell was taken up by cells at 5 hours (FIGS. 14A, B, C, and D). In general, shells are too big for cells to uptake.

Shells composed of (gelatin/PDDA)₅, 50 nm silica, and PDDA were successfully fabricated and observed under scanning electron microscope. Adding lipid bilayers on polymer shells was also demonstrated under fluorescence confocal microscope. Release of 80% DOX from shells took about 6 days. The 50% of drug was achieved after 8 hours. Cell cytotoxicity has shown that the strongest cytotoxicity was from free DOX solution. DOX-loaded shells were less toxic compared to free DOX solution. Empty shells did not show obviously cytotoxic effects in the cell culture study.

### EXAMPLE III

Cationic polymers used include poly(dimethyldiallyl ammonium chloride) (PDDA, MW 200 kD, Aldrich), poly(ethyleneimine) (PEI, MW 25 kD, Aldrich; MW 1.2 kD, Polysciences, Inc.), poly(allylamine) hydrochloride (PAH, MW 10 kD, Aldrich), and poly-L-lysine (PLL, MW 30 kD, Sigma). Negatively charged materials, including bovine albumin (MW 66kD, Sigma), gelatin (MW 50 kD-100 kD, Sigma), and poly(styrenesulfonate) (PSS, MW 70 KD, Aldrich), were selected for the LbL self-assembly. 1,2-Dipalmitoyl-sn-glycero-3-phosphocholine (DPPC) and 1,2-Dipalmitoyl-sn-glycero-3-phosphate (DPPA) (Avanti Polar Lipids, Inc) were prepared to form negatively charged liposomes. Copolymers PEI poly(ethyleneimine) 25K-poly(ethylene glycol) 5K (PEI-PEG) (1:1, 1:5, and 1:10) were synthesized and used for coating the outermost layer. Negatively charged 50 nm Fluoresbrite® YG Carboxylate nanoparticles (Polysciences, Inc) were used as a fluorescent label in the polymer multilayers for the polyelectrolyte shells. Weakly crosslinked melamine formaldehyde (MF) particles (1 and 5 micron in diameter, Microparticles GmbH, Germany), were used as templates for self-assembly.

Syntheses of PEI-PEG copolymers:

The monomethoxypoly(ethylene glycol) (mPEG) was first activated by esterification with maleic anhydride as reported by Shuai et al. (Shuai), and then conjugated to PEI through the amidation reaction. Briefly, PEI and the activated mPEG were added to a flask equipped with a magnetic stirring bar. The reaction flask was immersed in a 50° C. oil bath, and then high vacuum was applied. The amidation progress was monitored by FTIR spectroscopy. When the carbonyl group from carboxylic acid was no longer detectable, the reaction was stopped. The product was dissolved in methanol, precipitated in diethyl ether, and then vacuum dried. The grafting ratio of the purified copolymer was calculated from the integral values of characteristic peaks of PEG (e.g. CH₃O— at ˜3.38 ppm) and PEI (—CH₂CH₂— at ˜2.65 ppm) in the 1H NMR spectrum. Controlling the amount of PEG in reaction led to PEI-PEG copolymers with grafting ratio including from 1:1, 1:5 to 1:10.

Polyelectrolyte Shell Fabrication:

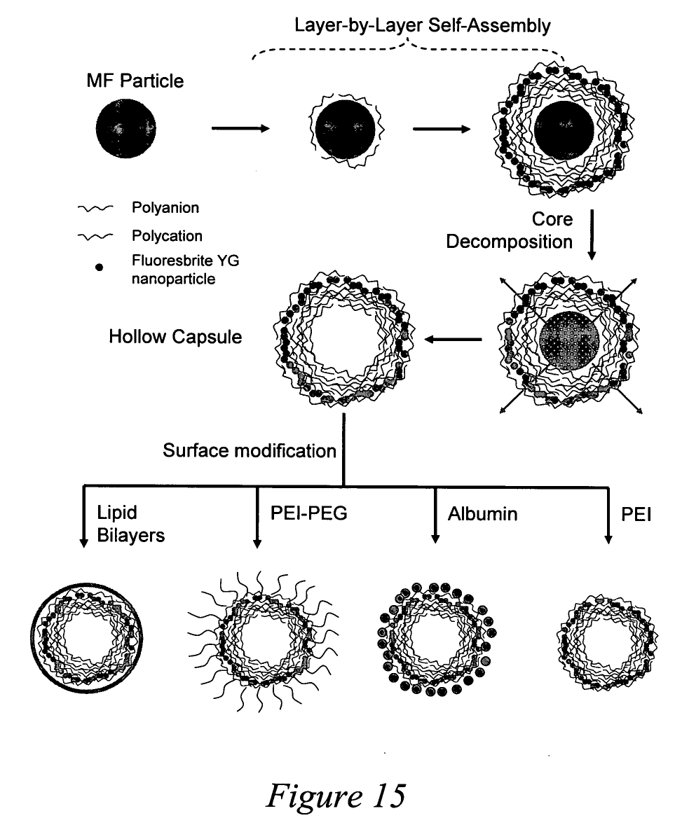

All polymer and protein solutions were prepared at the concentration of 2 mg/mL in pH 7.4 phosphate buffered saline (PBS). Gelatin and PDDA were used as a pair of oppositely charged polymers to build the initial layers on particle templates before the outermost layer was introduced. The shell fabrication procedure is illustrated in FIG. 15. First, MF microparticles (1 μm) were incubated in a gelatin solution for 40 minutes at room temperature. Centrifugation at 4,000 rpm for 5 minutes was used to remove free polymers in solution. The particle pellet was resuspended in deionized water and washed three times by centriftigation. A second layer of PDDA was introduced by following the similar coating process as described above. In total, five bilayers of gelatin and PDDA have been assembled on templates. Then another bilayer of FITC-labeled 50 nm nanoparticles (negatively charged) and PDDA were introduced. After the self-assembly, the coated particles were exposed in pH 1.2 HCl solution for 2 minutes to decompose the MF core. Hollow shells were obtained after washing with deionized water. We also repeated the above procedure with 5-micron MF particles to understand the effect of shell size on cell uptake. Generally, the common composition for all shells are (Gelatin/PDDA)₅+(nanoparticle/PDDA) and further layers could be added to achieve different surface charge and other properties.

To obtain a negative surface coating, PSS or albumin was directly adsorbed as the final outermost layer. To obtain a positively charged layer, coating of a negatively charged PSS layer is first introduced and then other cationic polymers (e.g., PEI or PLL) are adsorbed. PEI-PEG copolymers with different ratios were also assembled on a PSS layer. Liposomes (100 nm, 90% DPPC and 10% DPPA) were fabricated through mechanical extrusion. Final negatively charged lipid bilayers were assembled on hollow polymer shells through an established protocol (Moya). Polyelectrolyte shells were stored in Millipore H₂O (18.2 MΩ) for characterization purpose and stored in PBS for cell culture study.

Scanning Electron Microscopy:

Scanning electron microscopy (SEM) was used to characterize the size and morphology of polyelectrolyte shells. A drop of shell sample was dipped onto a flat mica surface and dried gently under nitrogen gas. A thin film of palladium with an approximate thickness of 2 nm was used to minimize electron charging on sample surface. Low voltage (2 or 5 KV) was used on a Hitachi S-4500 scanning electron microscope.

Zeta-Potential Measurement:

The zeta-potential of polyelectrolyte shells with different outermost layers was determined by using a zeta-potential analyzer (Zeta Plus, Brookhaven Instruments Corp). Three samples of each shell formulation were measured at 25° C. in 1 mM KCl solution. To understand how the shell surface charge will be altered by serum proteins, polyelectrolyte shells were also incubated in cell culture media (5% fetal bovine serum) for an hour and washed with deionized water before measurement.

Cell Culture:

Human breast cancer MCF-7 cells were obtained from ATCC, and seeded onto 6-well plates with a seeding density of 400,000 cells/well. Cells were maintained in Roswell Park Memorial Institute (RPMI) media supplemented with 5% heat-inactivated fetal bovine serum, 2 mM L-glutamine, 5,000 units/mL penicillin and 5 mg/mL streptomycin, 25 mM KCl, 25 mM D-glucose, and incubated at 37° C. in a humidified atmosphere with 5% CO₂. Cells were grown for one day before polymer shells were added. To examine any cytotoxicity that shells may induce during cell culture, a 3-day cell inhibition study was performed by a DNA assay (Labarca 1980). Cell uptake studies were also prepared in the conditions of serum-free environment to understand how the surface charge will play the role in cell uptake. Cells were seeded on the wells as the same conditions above for one day, and then culture media was removed and replaced with shell-containing HBSS solution and incubated for one hour.

Flow Cytometry:

Flow cytometry was used to quantitatively determine the percentage of cells containing internalized shells. Shells (4×107) suspended in a 2 mL culture media were added into each well and incubated for 4 and 24 hours. Then culture media including the free shells was removed and cells were detached by treatment with EDTA and trypsin. Cell pellets were obtained by centrifugation at 1200 rpm for 5 minutes and resuspended in PBS. To remove shells attached to the cell surface, cells were washed following a previous protocol (5 mM EDTA pH 5.0 for 15 min) (Behrens 2002). After filtration through a 50 micron Nylon mesh, cell suspension was analyzed by a Beckman Coulter Epics XL-MCL flow cytometer (15 mW Argon ion laser). Data analysis was conducted using a WinMDI program (Version 2.8).

Confocal Laser Scanning Microscopy:

Shells were incubated in cell culture wells for four hours before confocal laser scanning microscopy (CLSM) examination. To identify the shell location, cell nuclei and membranes were stained with Hoechst 33342 and lipophilic tracer DiI (Molecular Probes, Inc.) respectively. Samples were examined by CLSM using a Zeiss LSM 510 microscope (Zurich, Switzerland, lasers: He—Ne 543/633 nm, Ar 458/488/514 nm, and a tunable Ti-Sapphire laser (700-900 nm)) with a confocal plane of 300 nm. Z-sectioning was used for shell morphology study and identification of shell intracellular location. Image processing was performed on an IBM Graphics workstation using Zeiss LSM 510 software.

Characterization of shell morphology by SEM and CLSM:

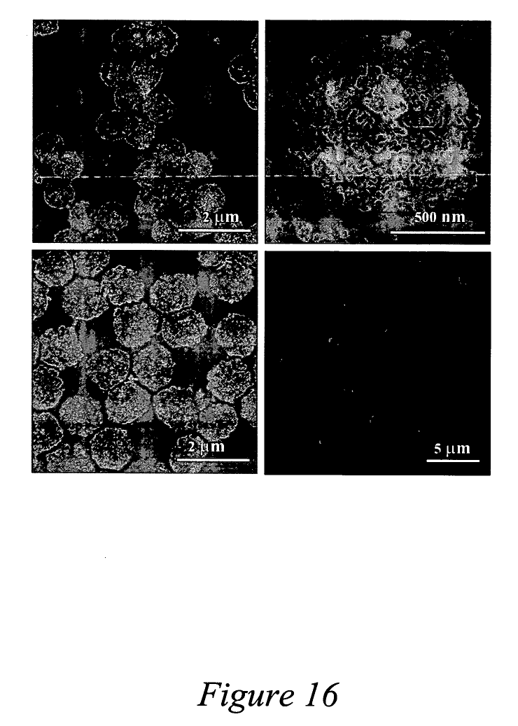

FIG. 2A shows the MF particles coated with multiple layers of (gelatin/PDDA)₅+(Fluoresbrite® YG Carboxylate nanoparticles/PDDA) before (FIG. 16A) and after acid dissolution (FIG. 16C) were compared through SEM investigation. All shells are not spherical but flat due to the removal of core and resulted in hollow capsules during drying process. Shell diameter is about 1.2 micron, which is larger than the 1-micron diameter of uncoated MF particles. FITC-labeled nanoparticles (50 nm) are clearly observed on the shell membrane (FIG. 16B). With the observation of different areas, most shells are individual and no obvious aggregation was found. Shells are well dispersed in PBS and remain spherical as confirmed from the reconstructured 3-D confocal images. FIG. 16D displays shell structure at a focal plane and similar to the SEM studies, most shells are single and few are aggregated. Shells are in green color due to the fluorescence from the FITC-labeled nanoparticles in the shell membrane. Shells are intact and the fluorescence is still preserved after storage at 4° C. in PBS for three months. Additionally, shells were incubated in serum-containing media for about four hours and no obvious aggregation was observed.

Shell Surface Charge and Cell Uptake:

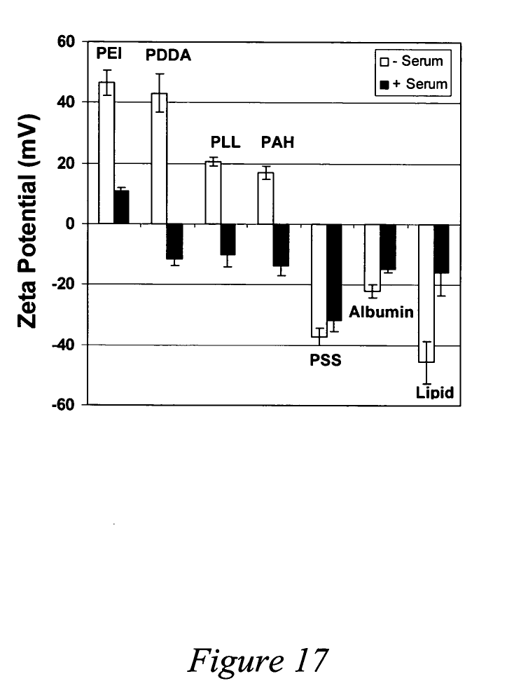

The outermost layer of a shell dominates the surface charge, which can be determined from zeta-potential measurement. Polycations (PDDA, PEI, PLL, and PAH) and polyanions (PSS, albumin, and lipid bilayers) covered shells were characterized by zeta-potential before and after one-hour culture media incubation to understand how serum protein adsorption on shell surface will modify the surface charge. Obviously, all shells having polycations on shell surface present positive charges and ranging from +17 to +47 mV (FIG. 17) before media incubation. Shells with outermost layers of PDDA and PEI present both strong positive charges of 43.1+6.4 mV and 46.5±4.1 mV due to the ammonium groups. PLL and PAH outermost layers are comparatively weakly charged with zeta-potential values of 20.6±1.4 mV and 16.9±2.1 mV. No statistical difference exists between PDDA and PEI layers but both of them have stronger positive charges than PLL and PAH layers (P<0.01). Between the two weakly charged positive layers, PLL layers are more positively charged compared to PAH layers (p<0.05).

After incubation of shells in cell culture media for one hour, all shell surface charges drop aggressively and three of them (PDDA, PLL, and PAH) present negative charges. The major proteins in the FBS are albumin and globulin with respective isoelectric points of 4.9 (Elmadhoun) and 5.0-5.1 (Chaiyasut; Anfinsen). Both of them are negatively charged at pH 7.4 and tend to interact with polycations such as PDDA, PEI, PAH, or PLL. PDDA, PLL, and PAH surface turned to negative charges of −11.6±2.3 mV, −10.3±3.8 mV, and −13.9±3.1 mV. Extended incubation up to six hours did not show more changes of the surface charges. This means equilibrium of the adsorption of proteins and other materials in serum onto shell membrane has already been reached after one hour, which is similar to previous studies. One possible reason for the PEI outermost layer shells maintaining the low positive charge of 10.8±1.2 mV may due to using branched polymers instead of linear polymers, with some amino groups not interacting with negatively charged proteins.

For shells covered with negatively charged materials, lipid bilayers present highest negative charges of −45.7±7 mV. PSS-covered shells have relatively strong negative charges compared to albumin but second to lipid bilayers. Sulfonate groups on PSS and phosphate groups on DPPA in the lipid bilayers contribute the strong negative charges. Adding albumin as an outermost layer gives a moderate negative surface charge (−22±2.1 mV) to the shell. The three different materials present three different negative charges (P<0.05) ranging relatively from high to low. All the negative charges were reduced after the one-hour incubation in culture media with zeta-potential values of −31.8±3.7 mV, −16±7.5 mV, and −14.7±1.2 mV corresponding to PSS, lipid, and albumin layers. The surface charges of an albumin layer and lipid bilayers change more dramatically than a PSS layer. Some positively charged materials might be absorbed onto those negative layers as indicated by the decrease of negative surface charge.

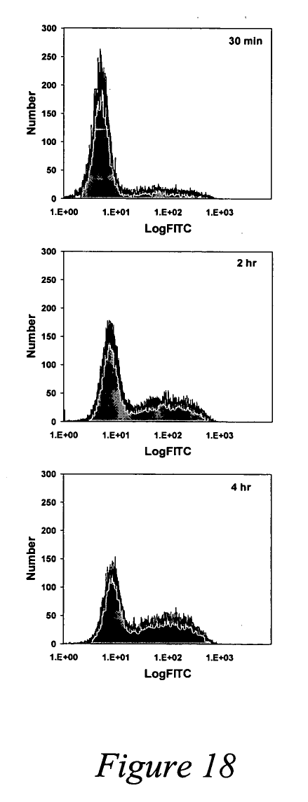

For LbL self-assembled shells, a surface charge can be easily adjusted by using different polycations and polyanions as outermost layers. To understand how surface charge corresponds to the degree of cell uptake particles, flow cytometry has been done for quantitative analysis. The data shown in FIG. 18 display two peaks and corresponding areas M1 and M2 were identified as cells with or without PEI covered shells. M2 represents the percentage of cells containing internalized shells. We also noticed that M2 region is increasing with time. At 30 minutes, about 18.9% cells have internalized shells. More cells have internalized shells after 2 and 4 hours shell-cell interaction studies corresponding to 43.6% and 51.7% respectively. Information on other shells uptake was obtained through this method.

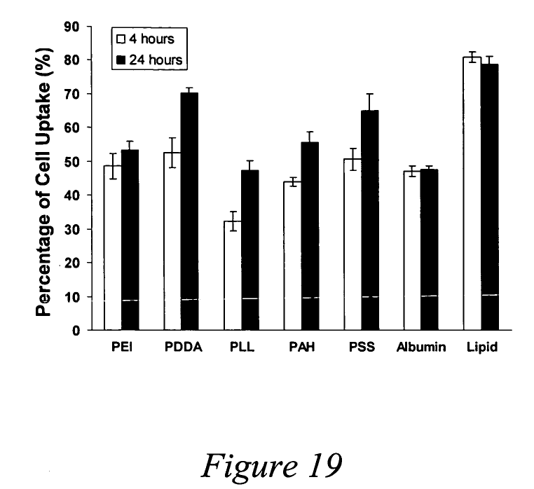

From the flow cytometry study, a positively charged shell surface does not necessarily mean a higher percentage of cell uptake. The highest percentage of cells with internalized shells is the group with the outermost layer of lipid bilayers (FIG. 19). About 80% cells were loaded with shells after the 4-hour shell-cell interaction study and no significant changes were noticed after 24 hours. The similar equilibrium was also found in the groups of albumin and PEI layers with a cell uptake of 47% and 49% at 4 hours. Other groups are showing significant cell uptake increase from 4 hours to 24 hours. For shells covered with polycations, PDDA has the highest cell uptake percentage of 70.3±1.6% and PLL has the lowest uptake percentage of 47.4±2.7% at 24 hours.

For shells with negative charges, a higher cell internalization corresponding to higher surface charges is noticed. Also, the negative charges of three materials in this study are not dependent carboxylate groups, so the surface charge is not the only controlling factor during shell uptake process. Shells covered with lipid bilayers showed the highest uptake percentage for 4 and 24 hours while albumin layers presented the lowest uptake.

To explore whether using neutral charged polymers such as PEG can reduce the material adsorption onto shell surface, copolymer PEI25k-PEG5k with different grafting ratios were synthesized for this purpose.

Shells covered with PEI-PEG copolymers

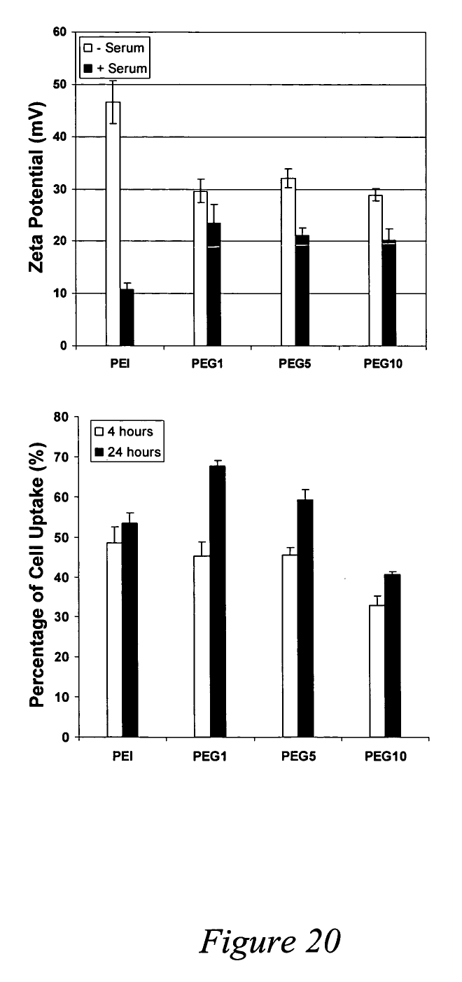

To understand how PEG effects shell surface charge and related cell uptake, copolymers PEI 25k-PEG 5k (1:1, 1:5, and 1:10) have been used as outermost layers for shell assembly. Even with the PEG polymer on shell surface, all of them present positive charges ranging from +30 mV to +32 mV (FIG. 20A). PEG is a hydrophilic polymer with neutral charge; the positive charge is due to the exposed amino groups on the PEI side chains, so the PEG portion of the copolymer could not dominate the surface charge, which is similar to previous reports of nanoparticles composed of PLA-PEG. All surface charges remained positive but decreased after incubation of shells in serum-containing media for one hour. Compared to PEI covered shells, the change of surface charge for PEI-PEG covered shells after serum incubation is much less (p<0.01). The shielding property of PEG is well known (Caliceti; Stone) and thus fewer negatively charged proteins were adsorbed on the shell surface compared to a PEI surface. Negatively charged proteins were adsorbed on PEI coated shells as indicated by the dramatic change of surface charge. In such a case, the amino group on PEI may not play a similar masking function as amidine groups.

For shells covered with PEI25k-PEG5k copolymers, a lower shell uptake percentage is related to a higher PEG grafting ratio (FIG. 20B). Shells covered with the PEI25k-PEG5k (1:10) copolymer resulted in the lowest cell uptake of 33% at 4 hours and 41% at 24 hours may be possibly due to the PEG's stealthy property, which has been widely reported (Auguste; Stone). Shells covered with other PEI25k-PEG5k copolymers (1:1 and 1:5) did not show the similar effect which may account for the relatively smaller number of PEG molecules on shell surface.

Shell-Cell Interaction with CLSM Study

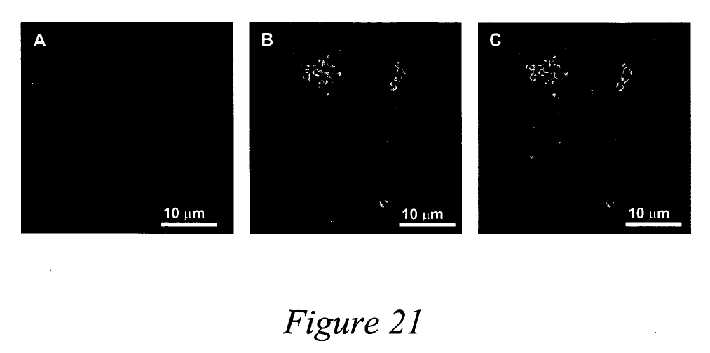

CLSM was used to qualitatively assess the shell uptake by cells. As shown from the confocal studies, most 1-micron shells with an outermost layer of PEI were internalized after four hours incubation while a few were still attached to the cell membrane (FIGS. 21A, B, and C). Internalized shells are all located in the cytoplasm and no shell was found in the nucleus. Since the shell size is much larger (>300 nm) for cell uptake through endocytosis, the internalization mechanism is believed to be phagocytosis, which was also suggested in other similar studies. Applicants have tested 5 micron shells covered with PEI, but no shells were uptake by cells even after 30 hours incubation. All shells were attached to the cell outer membrane.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. Polymeric nanoshells comprising two or more biocompatible polymer layers that define a hollow core, wherein the nanoshells are between 50 and 1000 nanometers in diameter and one or more of the polymer layers comprises charged organic polymers.

2. The polymeric nanoshells of claim 1, wherein the nanoshells have a diameter of between 50 and 600 nanometers.

3. The polymeric nanoshells of claim 2, wherein the nanoshells have a diameter of between 100 and 600 nanometers.

4. The polymeric nanoshells of any of claims 1, wherein the polymeric layer further comprises a magnetic resonance imaging contrast agent.

5. The polymeric nanoshells of any of claims 1, wherein the magnetic resonance imaging contrast agent is superparamagnetic iron oxide nanoparticles.

6. Nanospheres comprising polymeric nanoshells of any of claims 1, wherein the nanoshells are loaded with a bioactive agent.

7. The nanospheres of claim 6, wherein the bioactive agent is a therapeutic agent.

8. The nanospheres of claim 6, wherein the bioactive agent is a diagnostic agent.

9. Nanospheres comprising the polymeric nanoshells of claim 4, wherein the nanoshells are loaded with an antineoplastic drug.

10. Nanospheres comprising the polymeric nanoshells of claim 5, wherein the nanoshells are loaded with an antineoplastic drug.

11. The nanospheres of claim 7, wherein the therapeutic agent is an antineoplastic drug.

12. The nanospheres of claim 9, wherein the nanoshells further comprise a targeting moiety.

13. The nanospheres of claim 12, wherein the targeting moiety is selected from a peptide, a protein, a ligand, and an antibody.

14. The nanospheres of claim 13, wherein the targeting moiety is a tumor-specific antibody.

15. The nanospheres of claim 14, wherein the tumor-specific antibody is an anti-Her2/neu antibody.

16. The nanospheres of claim 11, wherein the antineoplastic drug is doxorubicin.

17. The nanospheres of claim 12, wherein the antineoplastic drug is doxorubicin.

18. The polymeric nanoshells of claim 1, wherein the shell surfaces have been modified by PEG.

19. The polymeric nanoshells of claim 18, wherein the shell surface comprises PEI25k-PEG5k (1:10).

20. A method of treating cancer, comprising administering to a subject in need thereof a composition comprising the nanospheres according to claim 9.

21. A method for making nanoshells for drug delivery, comprising
(a) contacting positively charged nanoparticle cores with a solution of a polyanion to form a coating of the polyanions on the nanoparticle cores;
(b) removing excess polyanion solution;
(c) contacting the resulting coated nanoparticles with a solution of a polycation to form a coating of the polycations on the nanoparticles;
(d) removing excess polycation solution;
(e) contacting the resulting coated nanoparticles with a solution of a polyanion to form a coating of the polyanions on the nanoparticles;
(f) removing excess polyanion solution, and
(g) dissolving the nanoparticle core;
wherein the nanoparticle cores are between 50 and 1000 nanometers in diameter and the polyanion and polycation are biocompatible and biodegradable organic polymers.

22. A method for making nanoshells for drug delivery, comprising
(a) contacting negatively charged nanoparticle cores with a solution of a polycation to form a coating of the polycations on the nanoparticle cores;
(b) removing excess polycation solution;
(c) contacting the resulting coated nanoparticles with a solution of a polyanion to form a coating of the polyanions on the nanoparticles;
(d) removing excess polyanion solution;
(e) contacting the resulting coated nanoparticles with a solution of a polycation to form a coating of the polycations on the nanoparticles;
(f) removing excess polycation solution; and
(g) dissolving the nanoparticle core,
wherein the nanoparticle cores are between 50 and 1000 nanometers in diameter and the polyanion and polycation are biocompatible and biodegradable organic polymers.

23. The method of claim 21, wherein (c) through (f) are repeatedly carried out, and optionally thereafter (c) and (d) are again carried out, so as to obtain a desired number of layers.

24. The method of claim 22, wherein (c) through (f) are repeatedly carried out, and optionally thereafter (c) and (d) are again carried out, so as to obtain a desired number of layers.

25. The method of claim 23, further comprising loading the resulting nanoshell with a drug substance.

26. The method of claim 24, further comprising loading the resulting nanoshell with a drug substance.

27. The method of claim 21, further comprising incorporating superparamagnetic iron oxide nanoparticles.

28. The method of claim 22, further comprising incorporating superparamagnetic iron oxide nanoparticles.

29. The method of claim 25, further comprising incorporating a targeting moiety into the outer shell.

30. The method of claim 26, further comprising incorporating a targeting moiety into the outer shell.

31. The method of claim 29, wherein the targeting moiety is an antibody.

32. The method of claim 30, wherein the targeting moiety is an antibody.

33. The method of claim 31, wherein the drug substance is an anti-neoplastic agent and the antibody is a tumor-specific antibody.

34. The method of claim 32, wherein the drug substance is an anti-neoplastic agent and the antibody is a tumor-specific antibody.
